(19) United States
(12) Patent Application Publication
(10) Pub. No.: US 2009/0082637 A1
(43) Pub. Date: Mar. 26, 2009

(54) MULTI-MODALITY FUSION CLASSIFIER WITH INTEGRATED NON-IMAGING FACTORS

(76) Inventor: Michael Galperin, Vista, CA (US)

Correspondence Address: FOLEY & LARDNER LLP, P.O. BOX 80278, SAN DIEGO, CA, 92138-0278, US

(21) Appl. No.: 11/859,311

(22) Filed: Sep. 21, 2007

(51) Int. Cl. A61B 5/00

(52) U.S. Cl. 600/300

(57) ABSTRACT

Disease or biomedical condition assessments or classifications are computed with scores from multiple different image modalities. Non-image information such as biometric, demographic, anthropomorphic and various risk factors may also be fused (combined) with one or more image modality disease or biomedical condition assessments or classifications to produce an integrated disease or biomedical condition assessment or suspicion score output and/or classification.

### BACKGROUND OF THE INVENTION

1. Field of the Invention

The invention relates to characterizing biomedical conditions, physical condition or disease using a variety of diagnostic or detection tools.

2. Description of the Related Technology

In the biomedical and clinical environment, a variety of image analysis systems have been proposed and developed to assist physicians in diagnosing disease from radiological images such as X-rays, MRI, mammography and ultrasound images. One example is U.S. Pat. No. 6,941,323 and U.S. Patent Publication 2005-0149360, both to Galperin et. al, and hereby incorporated by reference in their entireties. These documents describe an imaging system wherein an object in an image is compared to objects in other images to derive a measure of object similarity with further classification of the object in question based on measured similarities. If the object is a mass or lesion in a radiological image, it can be determined and/or assessed whether the object is more similar to malignancies or benign or masses in previously characterized studies.

Another example is U.S. Pat. No. 5,984,870 to Giger et al. In this patent, object similarities are not utilized. Instead, image features are numerically characterized, and an Artificial Neural Network (ANN) is statistically trained and used to derive a diagnosis for the image from the computed image features. This patent also discloses use of ANN pre-trained single classifier to derive a diagnosis from image features of the same lesion taken with different imaging modalities, such as both ultrasound and CAT scan. Although this is one possible approach to combining information from multiple imaging modalities to produce a single diagnosis, ANN have significant drawbacks. One is that they are subject to undertraining and overtraining and therefore prone to input-output data biases. Another is that their outputs are often not related to their inputs in an intuitive way (“black box” approach) that a physician would find useful in successfully using such a system in a real clinical environment.

Additional methods of enhancing image analysis to facilitate diagnosis or assessment of a condition would be beneficial in the field.

### SUMMARY

In one embodiment, the invention comprises a computer implemented method of producing a disease or condition assessment comprising producing a first numerical disease or condition classification score from at least one image, producing a second numerical disease or condition classification score from non-image information, combining at least the first and second disease or condition classification scores to produce a combined disease classification score, and displaying the combined disease classification score.

In another embodiment, a computer implemented method of producing a disease or condition suspicion (or assessment) classification score comprises producing a first numerical disease or condition suspicion (or assessment) classification score from at least one image produced with a first imaging modality, producing a second numerical disease or condition suspicion (or assessment) classification score from at least one image produced with a second imaging modality, combining at least the first and second disease or condition suspicion (or assessment) classification scores with non-neural network statistical analysis to produce a combined disease or condition suspicion (or assessment) classification score, and displaying the combined disease or condition suspicion (or assessment) classification score.

In another embodiment, a system for producing a disease or condition suspicion (or assessment) classification score comprises means for producing a first numerical disease or condition suspicion (or assessment) classification score from at least one image, means for producing a second numerical disease or condition suspicion (or assessment) classification score from non-image information, and means for combining at least the first and second disease or condition suspicion (or assessment) classification scores to produce a combined disease or condition suspicion (or assessment) classification score.

In another embodiment, a system for producing a disease suspicion classification score comprises means for producing a first numerical disease or condition suspicion (or assessment) classification score from at least one image produced with a first imaging modality, means for producing a second numerical disease or condition suspicion (or assessment) classification score from at least one image produced with a second imaging modality, and means for combining at least the first and second disease or condition suspicion (or assessment) classification scores with non-neural network statistical analysis to produce a combined disease or condition suspicion (or assessment) classification score.

### BRIEF DESCRIPTION OF THE DRAWINGS

FIG. 1 is a block diagram of a system that integrates classification information from multiple image modalities into a single suspicion or assessment score.

FIG. 2 is a flowchart of a method of image retrieval in one embodiment of the invention.

FIG. 3 is a block diagram of an image retrieval system according to the invention which may be utilized to carry out the method of FIG. 1.

FIG. 4 is a conceptual schematic of parameter sets associated with objects segmented from an image which may be created by the object parameterzation module of FIG. 3.

FIG. 5 is a flowchart of one embodiment of an object parameterization process which may be implemented in the object parameterization module of FIG. 2.

FIG. 6 is a screen display of user configured look up table filter functions according to one embodiment of the invention and which may be generated by the system of FIG. 3.

FIG. 7 is a screen display of user configured sharpening filter functions according to one embodiment of the invention and which may be generated by the system of FIG. 3.

FIG. 8 is a screen display of user configured general and edge enhancement filter functions according to one embodiment of the invention and which may be generated by the system of FIG. 3.

FIG. 9 is a screen display of user configured object definition according to one embodiment of the invention and which may be generated by the system of FIG. 3.

FIG. 10 is a screen display of user configured object searching and comparison according to one embodiment of the invention and which may be generated by the system of FIG. 3.

FIG. 11 is a screen display of user configured object searching, comparison and scoring similarity according to one embodiment of the invention and which may be generated by the system of FIG. 3.

FIG. 12 is a block diagram of a system that integrates classification information from one or more image modalities plus one or more non-image risk factors and physician classification input into a single suspicion score;

FIG. 13 is a block diagram illustrating integration of multiple image feature classifications into a single image modality classification;

FIG. 14 is a block diagram illustrating integration of multiple risk factor classifications into a single risk factor classification;

FIG. 15 is a block diagram illustrating integration of multiple image feature classifications plus one or more non-image risk factors and physician classification input information into a single image modality classification.

FIG. 16 is a screen display of a breast mammography image with a defined object which is assigned an LOS (Level of Suspicion) (also known as Computerized Lesion Assessment) (also known as Computerized Lesion Assessment) score of 3.7 based on comparison with template objects.

FIG. 17 is a screen display of a breast ultrasound image with a defined object which is assigned an LOS (Level of Suspicion) (also known as Computerized Lesion Assessment) score of 2.6 based on comparison with template objects.

FIG. 18 is a screen display of a breast MRI image with a defined object which is assigned an LOS (Level of Suspicion) (also known as Computerized Lesion Assessment) score of 2.0 based on comparison with template objects.

FIG. 19 is a screen display showing fusion of multiple imaging modalities and non-image factors for the lesion in FIGS. 16, 17, and 18 using modified integration filter.

FIG. 20 is a screen display illustrating a multimodality teaching file.

### DETAILED DESCRIPTION OF THE INVENTION

Embodiments of the invention will now be described with reference to the accompanying Figures, wherein like numerals refer to like elements throughout. The terminology used in the description presented herein is not intended to be interpreted in any limited or restrictive manner, simply because it is being utilized in conjunction with a detailed description of certain specific embodiments of the invention. Furthermore, embodiments of the invention may include several novel features, no single one of which is solely responsible for its desirable attributes or which is essential to practicing the inventions herein described.

As described above, it would be useful in a clinical environment to improve the contribution that automated image analysis can make to clinical screening and diagnosis. One way in which improvements can be made is by combining information from images of the same portion of the subject that are produced with different imaging modalities. Information from multiple image modalities can often provide an improvement in the accuracy of the disease likelihood score and resulting diagnosis. Different image modalities might include ultrasound, mammography, CT scan, MRI, and other imaging modalities currently known or to be developed (such as ultrasound tomography).

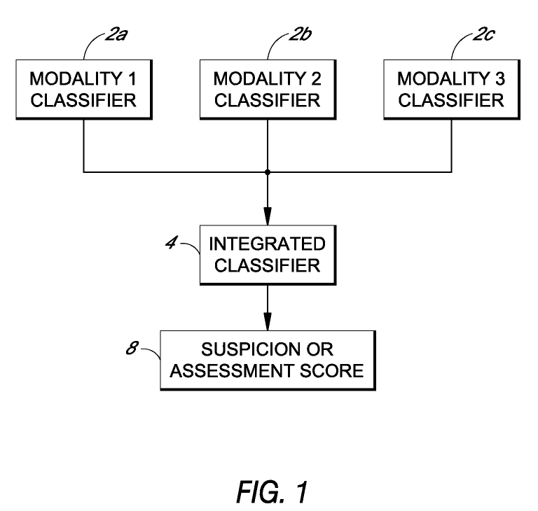

As shown in FIG. 1, this combination or fusion can be accomplished by combining classification or assessment scores produced by analysis of multiple imaging modalities. In FIG. 1, a classifier 2a analyzes one or more images to produce a disease likelihood score. Images from other imaging modalities are used in classifiers 2b and 2c to produce disease likelihood or assessment scores for other modalities. The numerical classifications from each of the multiple modalities are input to an integrated classification system 4 that combines the multiple numerical classifications into a single suspicion score 8. It will be appreciated that there is no limitation on the number of modalities that can be used. Any number from two or more can contribute to the integrated classification.

It is one novel and advantageous aspect of many embodiments of this system and method that the image analysis for each different modality is first separately distilled into single disease likelihood or assessment classification score prior to integration (fusion) by the integrated classification system 4. This is in contrast to techniques that may utilize a large number of individual image features from multiple image modalities (e.g. mass, aspect ration, density, texture, etc.) as inputs to an Artificial Neural Network that then produces a single output statistically averaged score of the trained classifier. As mentioned above, systems such as these are difficult to train without a bias, and the output is generally such a complex function of the inputs that intuitive relationships between the input information and the output score are lost and cannot be utilized to the utmost advantage (“black box” approach). Additionally any ANN assumes existence of “golden model” or “golden template” of targeted object. It is hypothesized by ANN developers that if trained properly and accurately the trained ANN will produce 100% accuracy in classification or recognition. Needless to say that such hypothesis is not realistic in the applications where the “golden model” is cancerous tumor for which a template simply does not and can not exist.

One advantageous score fusion method that avoids these problems is described in detail below. In all the herein described embodiments, the final score is advantageously displayed on a display device or otherwise output or transmitted to a physician, technician, or other party for review to assist in diagnosis and clinical decision making.

Before describing further methods of score fusion, advantageous individual modality assessment score computations will be described. The suspicion or assessment score for each individual modality may be calculated in a variety of ways. Described in detail below is an object definition and comparison method that the applicant has previously developed that has been found advantageous for producing suspicion or assessment scores for several different imaging modalities. It may also be noted that in some clinical practices multiple views of the same object (i.e. breast lesion) are assessed and scored. In some such cases each individual score of the each selected object view will be computed and then combined using rather non-statistical and non-mathematical clinical or practice guide. For example, in diagnostic breast ultrasound at least two views of a lesion in question (two views of the same object) will be assessed and scored by the radiologist as mandated by the practice guidelines. Then the score with the highest assessment of likelihood to malignancy will be selected as dictated by the regulated by the FDA guidance. In at least some such specific cases, scores from multiple views of the same lesion will not be subject to a fusion classification method because of the mandated practical guidelines but will be selected in accordance with the guidance and then integrated into the fusion classification process.

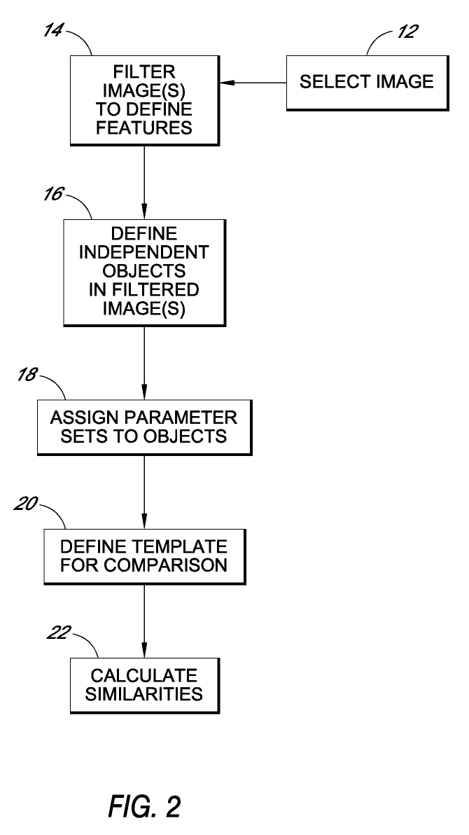

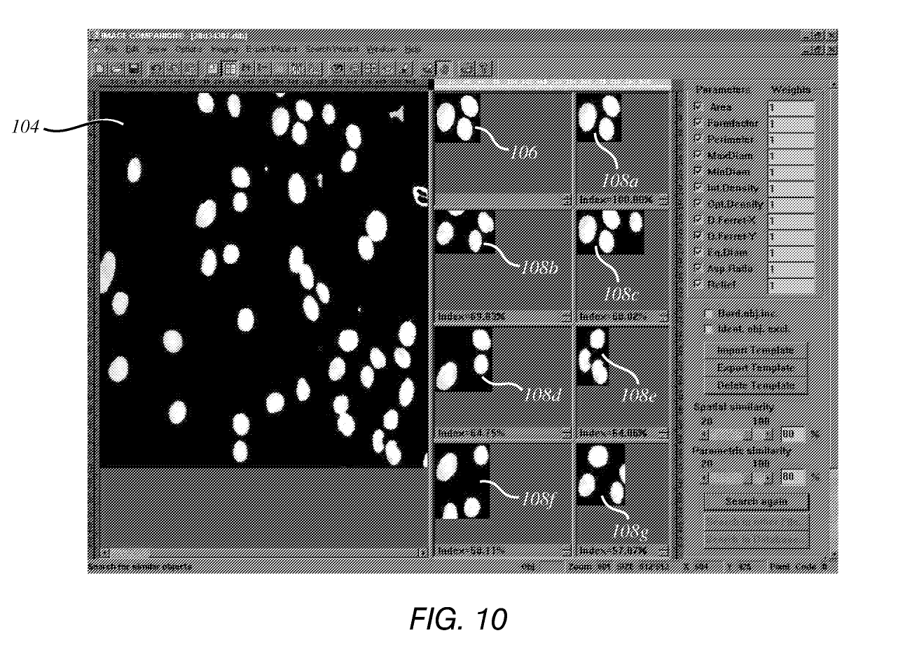

FIGS. 2-11 illustrate some specific advantageous methods of producing assessment scores which may be used in the modality fusion methods described herein. These methods generally start by comparing objects in a query image with objects in other images having known diagnoses.

Referring now to the flowchart of FIG. 2, a method of image comparison according to one embodiment of the method begins at block 12, where a starting or query image is selected. The query image will typically be provided by a user of the system and will comprise an image which contains one or more structures or objects of interest. Initially, the structure of interest in the image may not be well defined or distinct relative to the background. For example, the object boundaries may be poorly delineated, or it may have significant internal features present that are not immediately apparent in the image.

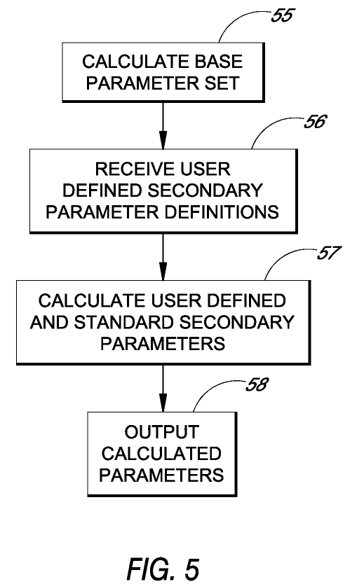

To help define the object of interest, both in terms of its boundaries and its internal features, the system performs image filtering at block 14. In advantageous embodiments, the filtering performed is under the control of the system user. The system may also perform filtering automatically using default filter functions or filter functions previously defined and stored by a user. A wide variety of well known image filtering techniques may be made available to the user. Many image filtering techniques which may be used in embodiments of the invention are described at pages 151-346 of The Image Processing Handbook, 2d Edition, John C. Russ, author, and published in 1995 by CRC Press, which is hereby incorporated by reference into this application in its entirety. Several filters which are utilized in one embodiment of the invention are set forth below with reference to FIGS. 5-7. These filters may enhance edges, enhance the appearance of pixels in particular brightness ranges, stretch contrast in selected pixel brightness ranges, reduce noise, or perform any of a wide variety of pixel processing functions. It will be appreciated that the filtering performed at block 14 may comprise the sequential application of several individual pixel filtering functions. Advantageously, filtering performed in block 14 can result in the enhancement of features which are characteristic of objects of interest or objects within a certain class, etc., but which do not appear in other objects or in the image background.

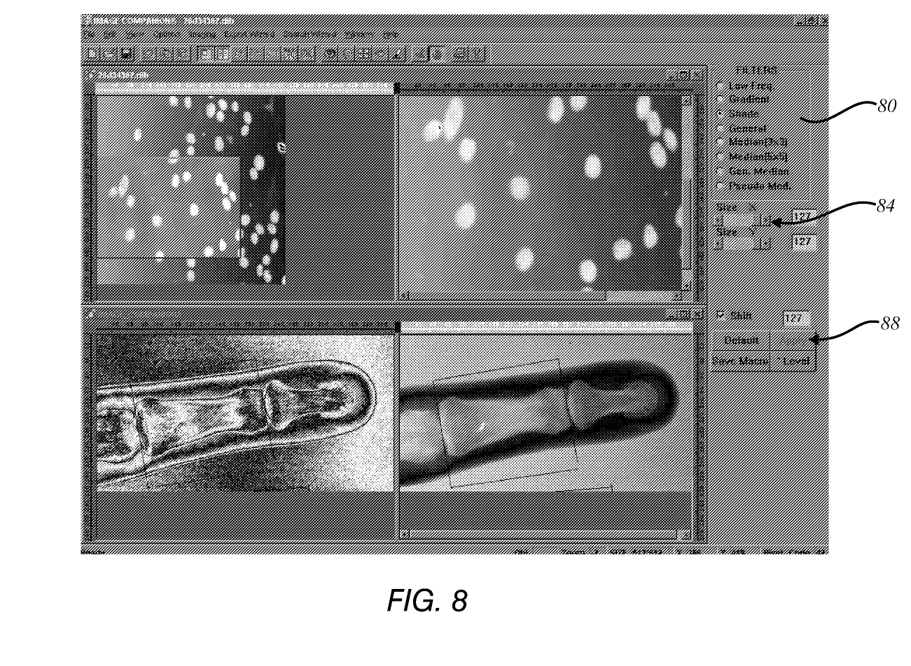

Following the filtering of block 14, objects within the filtered image are defined at block 16. Once again, this process may be performed under the control of the user, or performed automatically by the system. In general, this process involves evaluating pixel values so as to classify them as either an object pixel or a background pixel. As with the filtering performed at block 14, the object definition process of block 16 may be done using many well known techniques, some of which are described at pages 347-405 of The Image Processing Handbook mentioned above. Example object definition protocols provided in one embodiment of the invention are described in more detail with reference to FIG. 8.

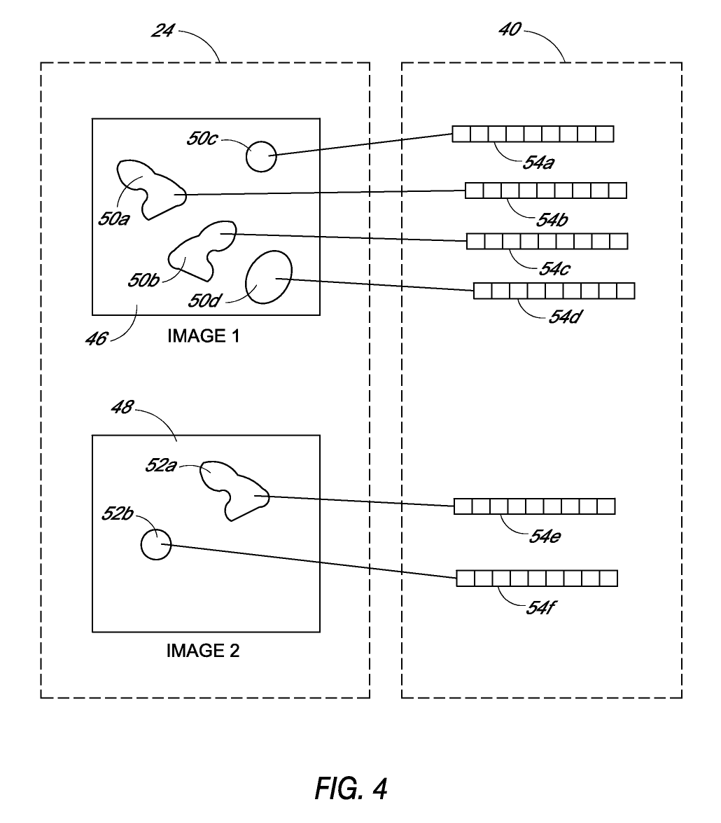

Next, at block 18, each defined object is separately numerically characterized by a set of parameters which are calculated from the pixel locations and brightness values of each defined object. In general, the numerical parameters are measures of the object's shape, size, brightness, texture, color, and other calculated characteristics. Preferably, the values present in the parameter sets are similar for objects of the same type. Example parameters which may advantageously be used in embodiments of the invention are described below with reference to FIG. 4.

Referring now to block 20, a template for comparison is defined by the user. The template may be a single defined object, or may be a group or cluster of defined objects in a region of the image. At block 22, similarities between the template and other objects or sets of objects are calculated. If the template is a single object, this may be done by comparing the parameter set assigned to the template object with the parameter sets assigned to other objects. There are several well known ways of evaluating the similarity between two parameter vectors. For example, Euclidean or Minkowski line metrics may be used. If the parameter set is represented as a bit string or in binary form (“present”—“absent”), the Hamming distance may be used as the similarity measure.

In certain embodiments of the invention, multi-dimensional non-binary parameter sets are associated with the objects, and as stated above, a comparison may be performed between not only individual parameter sets but also between parameter set groups associated with clusters of a plurality of objects. In this case, more complicated formulae have been developed and may be used, based on ideas set forth in Voronin, Yu. A., Theory of Classification and Its Applications 1985, published in Russia by Nauka. These formulae are set forth fully below. As is also explained below, if the template comprises a set of two or more objects, the comparison involves not only a comparison of the objects themselves, but also the spatial relationship between them. This method for numeric estimation of spatial relations between objects was developed by the inventors.

It will be appreciated that accuracy in identifying similar objects is improved when the filtering and object definition steps described above result in the enhancement of object features which are associated with objects of the desired class but not associated with objects not in the desired class. These enhanced features will manifest themselves as a numerically discriminable part of the parameter set, and the parameter set may thus be utilized to differentiate objects in the desired class from objects outside the desired class. Such differentiation manifested by the system using object border contour displays. The system may use different colors of the object border contours blue for objects touching the image edges, green—for allowed non-border objects, red—for objects filtered out by the system based on user set parameters intervals, and yellow—for template objects.

As one specific example, a query image may comprise a digital image of an area of skin pigmentation. A physician may be interested in evaluating the likelihood that the pigmentation in the image is a melanoma. Using a method according to the present invention, the digital image is filtered and an image area associated with the pigmentation is defined as an object within the image. Other images of skin pigmentation which are stored in an image database are also filtered and areas of skin pigmentation are defined as objects, advantageously using the same filters and object definition functions. These objects in the database are then also parameterized. The query parameter set is compared to the parameter sets associated with the database objects, and images of skin pigmentation which are similar are identified. Advantageously, the pigmentation area of the stored images have been previously characterized (diagnosed) as being melanoma or not. If retrieved similar object images are predominantly images of melanomas, the physician may be alerted that the possibility of melanoma for the query image is high. As mentioned above, it is advantageous if the filtering and object definition procedures enhance those aspects of skin pigmentation images which are closely associated with the presence of a melanoma. Furthermore, the parameter set itself may be tailored to the class of objects being analyzed. This may be done by assigning different weights to the different parameters of the parameter set during the comparison. For the melanoma example, a high weight may be assigned to parameters which are indicative of an irregular boundary or surface, while a lower weight may be assigned to a parameter associated with the total area of the object.

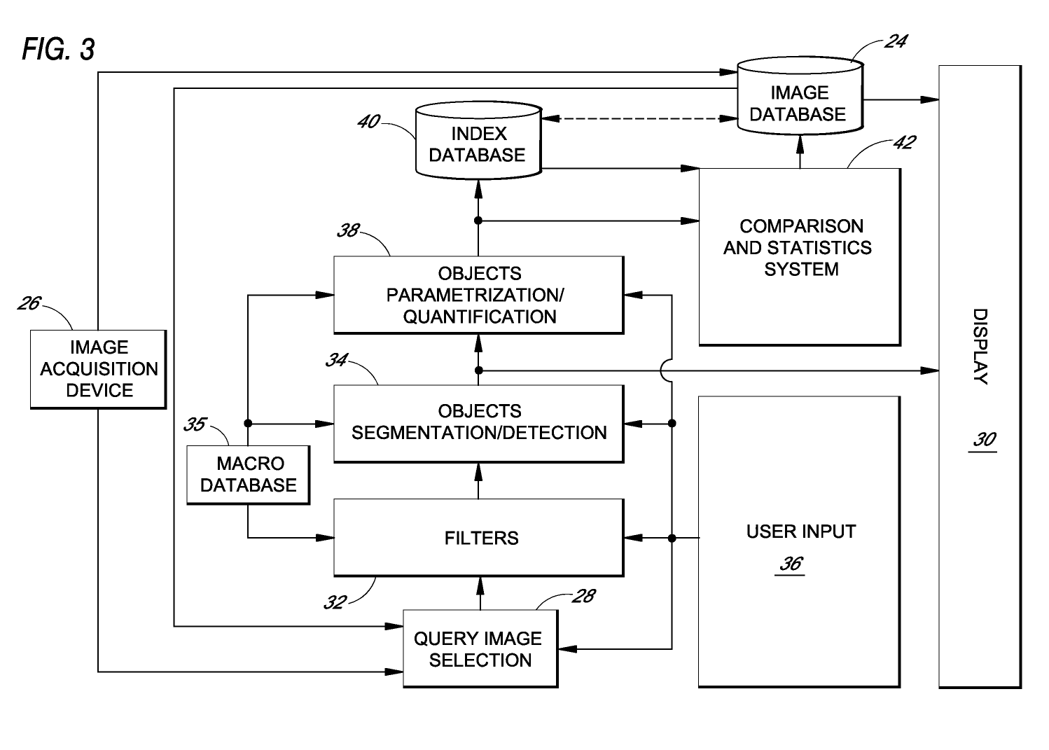

A system which may be used in one embodiment of the invention is illustrated in FIG. 3. An image acquisition device 26 is used to initially create images for storage in an image database 24 and/or for routing to a query image selection module 28 of the system. The image acquisition device may be a source of images of any type, including photographs, ultrasound images, X-ray or MRI images, a CRT display or trace, or any other data source having an output, which is definable as a collection of digital values. The image acquisition device may, for example, be a digital camera. The image acquisition device may produce the image directly. The system may also import previously created images from one or more imaging sources. The image acquisition device may be an external digital imaging source for such systems like PACS, RWS, LIS or the Internet or Telnet, for example. Typically, of course, the image data array processed by the system could be a two-dimensional array of pixels wherein each pixel is assigned an associated scalar or vector value. It is also well known that a two-dimensional array of pixels may be derived from a real 3D object that was represented by 2-dimensional “slices” or scans. For grey scale images, each pixel is associated with a brightness value, typically eight bits, defining a gray scale from zero (black) to 255 (white). 16-bit gray scale (0-4096 pixelcode level) or even 24-bit color formats are also used. For color images, a three component vector of data values may be associated with each pixel. The query image selection module, may, under the control of a user, select a query image from the image acquisition device, or may retrieve an image from the image database 24.

The system also comprises a display 30 which provides a visual output of one or more images to the user of the system. For example, the query image itself will typically be displayed to the user with the display device 30. This display of the query image may further be performed after image filtering by the filter module 32 and object definition by the object definition module 34. If no filtering or object segmentation has yet been implemented by the user with these modules, the unprocessed query image will be displayed to the user.

With a user input device 36 such as a keyboard, touchpad, or mouse, the user may control the filter module 32 so as to implement the filtering described above with reference to block 14 of FIG. 2. It is one aspect of some embodiments of the invention that the image continues to be displayed as the filtering is implemented. Thus, as the user modifies the filter function being performed by the filter module 32, the visual impact of the filter application on the image is displayed to the user.

The user may also control the implementation of object definition by the object definition module 34. Pixel brightness thresholds and other features of the object definition procedure may be modified by the user with the input device 36. As with the filtering operation, the image may be displayed after object definition so that the user can observe visually the contours and internal features of objects defined in the image. If the object definition technique is modified by the user, the display of the image may be accordingly updated so that the user can evaluate the effects of the filtering alterations and image object changes graphically on the display.

In some embodiments, the user may allow the system to perform object definition automatically, without requiring any additional user input. Of course, the above described display updates may be performed after this automatic object definition as well. As is also illustrated in this Figure and is explained further below with reference to FIG. 5, the user may also control aspects of parameter calculation via the user input device 36.

It will also be appreciated that in many applications, multiple images having similar sources and structures will be processed by the user in the same way (“batch processing”). For example, cranial X-ray images may all be processed with the same filter set and object definition functions prior to parameterization—in batch. This helps ensure that compatible images and objects therein are parameterized for comparison. Of course, care must be taken that the sources of the images are themselves compatible. Overall brightness, dimensional variations, and other differences between, for example, different microscopes used to obtain the query image and images in the database 24 should be compensated for either prior to or as part of the processing procedures, known as dimension and/or brightness calibration.

To facilitate this common processing of multiple images user defined macros of filter and object definition and detection functions may be stored in a macro database 35 for future use on additional images. The user-friendliness of the system is improved by this feature because images from similar sources can be processed in the same way without requiring the user to remember and manually re-select the same set of filtering and object definition functions when processing similar images in the future. In one embodiment, the user may operate on an image using either individual filter and object definition functions stored in the macro database or user defined groups of individual filter and object definition functions stored in the macro database 35.

The object definition module 34 is connected to an object parameterization module 38, which receives the pixel values and contour coordinates of the objects defined in the image. This module then calculates the parameter sets described above with reference to block 18 of FIG. 2 using the input pixel values. The calculated parameter sets may be stored in an index database 40 for future use. During the image searching, evaluating and retrieval process, one or more parameter sets associated with a template will be forwarded to a parameter set comparison module 42 along with parameter sets associated with other objects in the image or other objects in images stored in the image database 24. Objects or object clusters that are similar to the template, are then also displayed to the user on the display 30.

Referring now to FIG. 4, it is one aspect of the invention that any given image may have associated with it several different parameter sets, with each parameter set associated with a detected object in that image. Thus, the image database 24 may store a plurality of images 46, 48, each of which includes a plurality of defined objects 50a-d and 52a-b. Each object is associated with a parameter set 54a-f, which is stored in the index database 40.

In one embodiment, the parameter set includes a computation of the object area by a formula which counts the number of pixels defined as part of object “A” and multiplies that number by a calibration coefficient as follows:

$${\sum }_{i , j} z * {δ}_{ij} , {δ}_{ij} = \{ \begin{matrix}1 , & ij ∈ A \\ 0 , & ij ∉ A\end{matrix} , \qquad ( 1 )$$

where z is a user defined dimensional calibration coefficient.

When the object has many internal holes, the area parameter may be calculated instead by the formula:

$$\frac{{\sum }_{i} ( {X}_{i} + {X}_{i - 1} ) * ( {Y}_{i} - {Y}_{i - 1} )}{2} , \qquad ( 2 )$$

wherein X, Y are the coordinates of the periphery pixels of the object.

Other advantageous object characterization parameters include the length of the perimeter, and the maximum and minimum diameters of the object through the center of gravity of the object. These may be calculated with the formulas:

$${\sum }_{i} \sqrt{{( {X}_{i} - {X}_{i - 1} )}^{2} + {( {Y}_{i} - {Y}_{i - 1} )}^{2}} \qquad ( 3 )$$

for perimeter,

$$4 * \sqrt{\frac{\begin{matrix}\overline{{x}^{2}} - {\overline{( x )}}^{2} + \overline{{y}^{2}} - {\overline{( y )}}^{2} + \\ \sqrt{{( \overline{{x}^{2}} - {\overline{( x )}}^{2} - \overline{{y}^{2}} + {\overline{( y )}}^{2} )}^{2} + 4 * {( \overline{xy} - \overline{x} * \overline{y} )}^{2}}\end{matrix}}{2}} , \qquad ( 4 )$$

for maximum diameter, and

$$4 * \sqrt{\frac{\begin{matrix}\overline{{x}^{2}} - {\overline{( x )}}^{2} + \overline{{y}^{2}} - {\overline{( y )}}^{2} - \\ \sqrt{{( \overline{{x}^{2}} - {\overline{( x )}}^{2} - \overline{{y}^{2}} + {\overline{( y )}}^{2} )}^{2} + 4 * {( \overline{xy} - \overline{x} * \overline{y} )}^{2}}\end{matrix}}{2}} , \qquad ( 5 )$$

for minimum diameter, where

$$\overline{x} = \frac{( {\sum }_{j , i ∈ A} {X}_{ij} )}{( {\sum }_{j , i ∈ A} {δ}_{ij} )} , \\ \overline{y} = \frac{( {\sum }_{j , i ∈ A} {Y}_{ij} )}{( {\sum }_{j , i ∈ A} {δ}_{ij} )} , \\ \overline{{x}^{2}} = \frac{( {\sum }_{j , i ∈ A} {X}_{ij}^{2} )}{( {\sum }_{j , i ∈ A} {δ}_{ij} )} , \\ \overline{{y}^{2}} = \frac{( {\sum }_{j , i ∈ A} {Y}_{ij}^{2} )}{( {\sum }_{j , i ∈ A} {δ}_{ij} )} , \\ \overline{xy} = \frac{( {\sum }_{j , i ∈ A} {X}_{ij} * {Y}_{ij} )}{( {\sum }_{j , i ∈ A} {δ}_{ij} )}$$

Other shape and size related parameters may be defined and included in the parameter set, such as form factor:

$$\frac{4 * π * Area}{{( Perimeter )}^{2}} \qquad ( 6 )$$

equivalent circular diameter:

$$\sqrt{\frac{4 * Area}{π}} \qquad ( 7 )$$

and aspect ratio, which represents the ratio of the maximum diameter and minimum diameters through the center of gravity. The maximum and minimum Ferret diameters of the object may also be included as part of the parameter set, namely:

maxXᵢⱼ−minXᵢⱼ;max Yᵢⱼ−minYᵢⱼ, (8)

where

i,jεA

Parameters which relate to pixel intensities within the object are also advantageous to include in the object characterization parameter set. These may include optical density, which may be calculated as:

$$- {log}_{10} ( \frac{\frac{{\sum }_{ij ∈ A} {I}_{ij}}{{\sum }_{ij ∈ A} {δ}_{ij}}}{{I}_{max}} ) \qquad ( 9 )$$

and integrated density:

$${\sum }_{i , j ∈ A} {I}_{ij} \qquad ( 10 )$$

where Iᵢⱼ is the brightness (i.e. 0-255 for 8-bit images or 0-65536 for 16-bit images or 0-16777216 for 24-bit images) of pixel ij, and Iₘₐₓ is the maximum pixel brightness in the area/image.

More complicated intensity functions which parameterize the texture of the object may be utilized as well. One such parameter is a relief parameter which may be calculated as:

$${\sum }_{i , i ∈ A ; Nij \geq 2} {rl}_{ij} / {\sum }_{i , j ∈ A ; Nij \geq 2} {δ}_{ij} , \\ where \\ {rl}_{ij} = {r}_{ij} * Ω ( Nij ) ; where Ω ( {N}_{ij} ) is a function of {N}_{ij} \\ {r}_{ij} = ( {\sum }_{m = i - 1}^{i + 1} {\sum }_{n = j - 1}^{j + 1} abs ( {l}_{nm} - {l}_{ij} ) ) / {N}_{ij} ; n , m ∈ A ; \\ {N}_{ij} = {\sum }_{n = i - 1}^{i + 1} {\sum }_{m = j - 1}^{j + 1} {δ}_{nm} \qquad ( 11 )$$

This parameter belongs to a textural class of parameters and is a measure of the average difference between a pixel values in the object and the values of its surrounding pixels. In the simplest case, Ω(Nᵢⱼ)=Nᵢⱼ, although the function may comprise multiplication by a constant, or may involve a more complicated function of the number of nearest neighbors or pixel position within the object.

Other examples include homogeneity:

$$Φ = {\sum }_{Ii} {\sum }_{Ij} {( {N}_{ij} / \overline{N} ( {DiameterFerret}_{{x}_{y}} ) )}^{2} , \qquad ( 12 )$$

where I is intensity; i, jεA; and N̅ is a renormalizing constant and contrast:

$$L = {\sum }_{{Ii}_{ } - Ij = 0} {( {I}_{i} - {I}_{j} )}^{2} [ {\sum }_{Ii - Ij} ( {N}_{ij} / \overline{N} ( {DiameterFerret}_{xy} ) ) ] , \qquad ( 13 )$$

where I is intensity; i, jεA; and N̅ is a renormalizing constant

It will be appreciated that the nature of the parameter set may vary widely for different embodiments of the invention, and may include alternative or additional parameters not described above. The parameters set forth above, however, have been found suitable for object characterization in many useful applications.

FIG. 5 illustrates a flowchart of the parameter set generation process which may be performed by the object paramterization module 38 of FIG. 3. Initially, at block 55, the base or fundamental parameters are calculated. These are the parameters that use raw pixel positions or intensities as inputs. Examples include area (Equation 1), perimeter (Equation 3), integrated intensity (Equation 10), etc. Another set of parameters, referred to herein as “secondary” parameters are also calculated. These are parameters which are functions of the base parameters, and which do not require any additional pixel specific information for their calculation. Examples of standard secondary parameters include Formfactor (Equation 6) and aspect ratio. In some embodiments, the user is allowed to define additional secondary parameters for object characterization which may have significance in certain image analysis applications. For example, a new hypothetical parameter comprising the ratio of Formfactor to Area may be defined and made part of the object characterization parameter set. Thus, at block 56, the system may receive user input (by entering information into a dialog box with a mouse and/or keyboard, for example) regarding secondary parameter definitions not already utilized by the system.

At block 57 the system calculates both the user defined and standard secondary parameters, and at block 58 the parameters thus calculated are formatted into a feature vector and output to either or both the index database 40 and the comparison and statistics system 42 of FIG. 3.

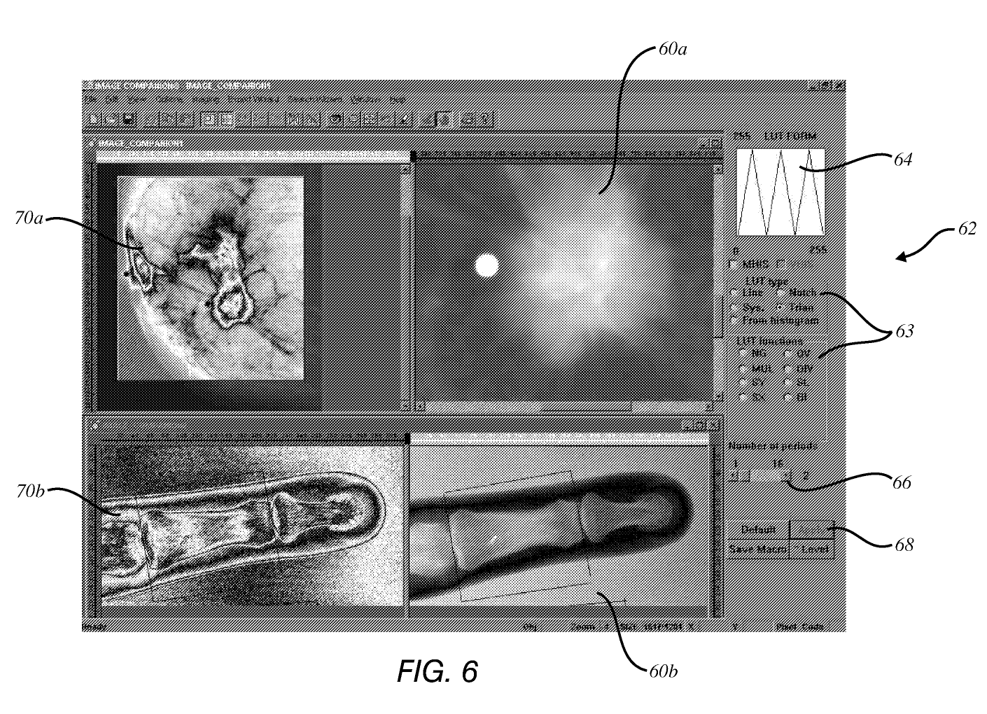

In FIGS. 6 through 10, a specific implementation of the invention is illustrated by example screen displays which illustrate aspects of user control (via the input devices 36 of FIG. 3) and visualization (via the display 30 of FIG. 3) of the filtering and object definition processes. As will be apparent to those of skill in the art, this embodiment of the invention is implemented in software on a general purpose computer. A wide variety of data processing system environments may be utilized in conjunction with the present invention. In many embodiments, the invention is implemented in software coded in C/C++ programming languages and running on a personal computer or workstation with suitable memory in the form, for example, of RAM and a hard drive. The computer in this implementation will typically be connected to an image database through a local or wide area network, or via PACS, RIS, LIS or Internet/Telnet client-server system using standard methods of communications such as direct input/output or DICOM Server. In another implementation, the computer runs a standard web browser, which display a communicating application and accesses image databases and image analysis and computer-aided detection software hosted on a remote Internet server. In these embodiments, the web tier may comprise ASP program files that present dynamic web pages. A middle tier may comprise a .NET components wrapper to the API library and ADO.NET “accessory” to the database. The data tier may comprise the database of sessions and pointers to image files in the data server. An image grid control module which displays users saved session images may use control and thumbnail generator components. These components in turn may access the session data residing in the data server, as well as the image files saved in the file system. Standard DICOM protocol and server communication may be implemented. The web application of the multimodality fusion system described further below may be logically layered into three tiers for each modality. Then one additional integrated layer may be implemented for the fusion classification.

An Intranet version of the application is also envisioned and implemented. In such case the system works as a part of PACS, for example, using LAN and HIS as a hosting system.

Referring now to FIG. 6, original images 60a and 60b are displayed to the user of the system in respective portions of the display. The upper display 60a comprises a close up of a suspected malignancy in a mammogram. The lower display 60b is a bone density image utilized in evaluating osteoporosis. On another portion 62 of the screen is a display of a filter protocol. This portion 62 of the screen display shown one of the computationally simplest filtering techniques under user control in this embodiment, which is look-up-table (LUT) filtering. With this filter, each input pixel brightness value is mapped onto an output pixel brightness value. If pixel brightness ranges from a value of 0 (black) to 255 (white), each value from 0 to 255 is mapped to a new value defined by the LUT being used.

In this embodiment, the user is provided with a visual indication 64 of the look-up table form being applied, with input pixel values on the horizontal axis and output pixel values on the vertical axis. Using user selectable check boxes 63, the user may define the nature of the look-up-table filter being applied. In this embodiment, the user may define both a table form and a table function. The form may be selected between linear (no effect on pixel values), triangular, and sawtooth (also referred to as notch). The triangular form is illustrated in FIG. 6. For the triangular and sawtooth forms, the user may be provided with a slidebar 66 or other input method for selecting the number of periods in the input brightness range. The user may also import a previously used user defined LUT if desired.

The look-up-table form may also be varied by additional user defined functions. These functions may include negative inversion, multiplication or division by a constant, binarization, brightness shifting, contrast stretching, and the like. For each of these functions, the user may control via slidebars or other user manipulatable displays the constants and thresholds utilized by the system for these functions. Histogram based look-up table filtering may also be provided, such as histogram equalization and histogram based piecewise contrast stretching. After the user defines the desired LUT filter, they may apply it to the image by selecting the “APPLY” button 68. The look-up-table defined by the user is then applied to the image or a selected portion thereof.

Furthermore, second display 70a and 70b of the image is provided following application of the three period triangular LUT filter. If the user modifies the LUT filter function, the image display 70a, 70b is updated to show the visual result of the new filter function when the user clicks the APPLY button 68. Thus, the user may view a substantially continuously updated filtered image as the filter functions used are modified. In filtered image 70a, regions of suspected malignancy are enhanced with respect to the background following LUT application. In the filtered image 70b, the bone density variations present in the central bone segment are enhanced and pronounced.

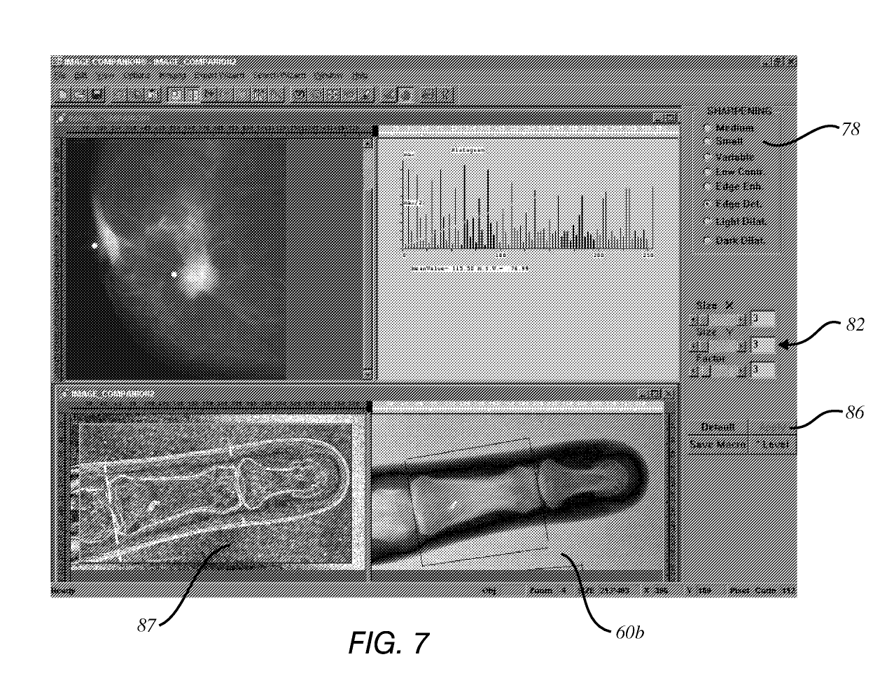

In addition to LUT filtering, convolution filters, frequency domain filters, and other filter types may be utilized to further enhance and define significant features of imaged objects. Several specific examples provided in one embodiment of the invention are illustrated in FIGS. 7 and 8. In analogy with the user interface for the LUT filtering described with reference to FIG. 6, additional filter types may be selected with checkboxes 78, 80. Filter parameters such as filter box size are user controllable via slidebars 82, 84. APPLY buttons 86, 88 initiate the filter operation and display update to show the filtered image or image region. In FIG. 7, the bone image 60b is filtered with a 3×3 edge detection filter which produces the filtered image 87 having enhanced pixels along edges in the image. In FIG. 8, a region of interest 89 in an image of blood cells in bodily fluids where a shading filter was used to compensate for a background brightness variation across the image.

In the specific implementation illustrated in FIGS. 7 and 8, the following base set filter functions may be applied by the system user:

1. Sharpening of Small Size Details on Image

This type of filter belongs to a class of Laplacian filters. The filter is a linear filter in the frequency domain. The 3×3 kernel is understood to mean that central pixel brightness value is multiplied by 4. As a result of this filtering, the sharpness of small details (not to exceed 3×3) of the image is increased.

$${C}_{mn} = \{ \begin{matrix}- 1 & - 1 & - 1 \\ - 1 & 9 & - 1 \\ - 1 & - 1 & - 1\end{matrix} \}$$

2. Sharpening of Middle Size Details on Image

This type of filter belongs to a class of Laplacian filters. Functionality is similar to the 3×3 kernel type filter. As a result of this filtering, the sharpness of small details (not to exceed 5×5) of the image is increased.

$${C}_{mn} = \{ \begin{matrix}- 1 / 12 & - 1 / 12 & - 2 / 12 & - 1 / 12 & - 1 / 12 \\ - 1 / 12 & - 2 / 12 & 3 / 12 & - 2 / 12 & - 1 / 12 \\ - 2 / 12 & 3 / 12 & 28 / 12 & 3 / 12 & - 2 / 12 \\ - 1 / 12 & - 2 / 12 & 3 / 12 & - 2 / 12 & - 1 / 12 \\ - 1 / 12 & - 1 / 12 & - 2 / 12 & - 1 / 12 & - 1 / 12\end{matrix} \}$$

3. Sharpening of a Defined Size Details on Image

This filter performs convolution transformation of the image through a user defined multiplication factor. As a result, all details of a user defined size are sharpened. The size of processed image detail may be defined through available editing submenu windows for X and Y dimensions.

$${I}_{out} = {I}_{in} * ϑ * ( {I}_{in} - {\sum }_{Ω} {I}_{in} / ( m * n ) ) , \\ \begin{matrix}where ϑ is the user defined multiplication \\ fact or and Ω is the mxn filter box\end{matrix} \qquad ( 14 )$$

4. Sharpening of a Low Contrast Details

This filter performs convolution transformation of the image and belongs to a spatial domain filters. The filtering is performed through a user defined multiplication Factor and automatically calculated special parameter. This parameter is a ratio of a current pixel value to Mean Square Deviation of a pixel value calculated for the given size of the pixel aperture (or filter box). As a result, all details of a user defined size are sharpened. The size of the processed image detail may be defined through available for editing submenu windows for X and Y dimensions.

$${I}_{out} = {I}_{in} * ϑ * μ * ( {I}_{in} - {\sum }_{Ω} {I}_{in} / ( m * n ) ) , \\ where ϑ is factor and μ is ( {\sum }_{Ω} {I}_{in} / ( m * n ) ) / {σ}_{Ω} \qquad ( 15 )$$

5. Edge Enhancement Filter

This edge enhancement filter belongs to a non-linear range filter. User defines the size of the filter box. This filter provides two regimes, selected by the user. If the default regime Strong is changed by the user to regime Weak, the filter will change the processing method to avoid images noise impact in certain high frequencies.

Iₒᵤₜ=Sup_{Ω},when Iᵢₙ>½*(Sup_{Ω}+Inf_{Ω})

Iₒᵤₜ=Inf_{Ω},when Iᵢₙ≦½*(Sup_{Ω}+Inf_{Ω}) (16)
where Sup_{Ω} is maximum brightnesss within filter box and Inf_{Ω} is minimum brightness within filter box

6. Edge Detection

This edge detection filter belongs to modified Laplacian omnidirectional edge detection convolution filters. User defines the size of the filter box. This filter performs edge detection of the image through a user defined Factor. The Factor is used for convolution mask values calculations

7. Dilation filters

Both filters belong to morphological class and are inversive to each other. The first one should be used for image light elements dilation, the second one—for dark elements dilation. If the default regime Strong is changed by the user to regime Weak, both filters will change the processing method to avoid images noise impact in certain high frequencies. In general:

Iₒᵤₜ=Sup_{Ω} or Iₒᵤₜ=Inf_{Ω} (17)

8. Low Frequency

This filter represents a convolution transformation of modified Gaussian type. It belongs to a class of linear filters in frequency domain. The size of pixel box or aperture is defined by the user for X and Y dimensions. The filter is used often for certain frequencies noise reduction. In general:

$${I}_{out} = ( {\sum }_{Ω} {I}_{in} / ( m * n ) ) \qquad ( 18 )$$

9. Gradient/Modified Sobel Edge Detection Filter

This filter belongs to a non-linear edge-detection class. The filter uses a technique with partial derivatives replacement with their estimates. It is known in image processing as a Sobel filter. The size of the pixel box or aperture defined by the user for X and Y dimensions. This filter performs convolution transformation of the image through a user defined amplification Factor. The user also is provided with the ability to set a binarization Threshold if a correspondent check-box is marked. The threshold serves as a modification to the classic Sobel filter and enables the user to find right flexibility for the edge detection process. If the threshold is used the outcome of transformation will be a binary image. The default but modifiable masks are:

$${C}_{mn} = \{ \begin{matrix}1 & 0 & - 1 \\ 2 & 0 & - 2 \\ 1 & 0 & - 1\end{matrix} \}$$

$${C}_{mn} = \{ \begin{matrix}- 1 & - 2 & - 1 \\ 0 & 0 & 0 \\ 1 & 2 & 1\end{matrix} \}$$

10. Shading Correction

This filter belongs to a smoothing class filter. The size of the pixel box or aperture is defined by the user for X and Y dimensions. The filter is modified from a classical type shading correction filter by enabling the user with shifting capability. If check-box Shift is marked the user will be able to change the default value of the shift to a custom one. This filter is very handy for elimination of a negative lighting impact which sometimes occurs during the image acquisition process.

$${I}_{out} = ( {I}_{in} - {\sum }_{Ω} {I}_{in} / ( m * n ) ) + Shift , \\ where Shift dy default is 127 \qquad ( 19 )$$

11. General or Universal Filter

This is a convolution type filter with a user controlled size of the kernel and the weights mask values. The default size of the kernel is 9×9. For the user's convenience, the convolution mask contains default typically used weights values. Push-button activates the customization regime when the user is able to modify dimensions of the mask and then modify default weights in the convolution mask.

12. Median (3×3) filter

Moving median (or sometimes referred as rank) filter produces as an output the median, replacing a pixel (rather than the mean), of the pixel values in a square pixel box centered around that pixel. The filter is a non-linear type filter with the filtration window dimensions of 3×3. Usually used to eliminate very small details of the image sized at 1-2 pixels.

13. Median (5×5) Filter

Similar to the filter described above, but with the filtration window dimensions 5×5. Usually used to eliminate small details of the image sized at up to 5 pixels.

14. General Median Filter

This filter is similar to the filters described above, but with the filtration window dimensions set by the user. The size of eliminated details depend on the size of the set filtration window.

15. Psuedomedian Filter

This filter is similar to median type filters described above. However it provides rectangular filtration window controlled by the user and performs transformation in a two pass algorithm.

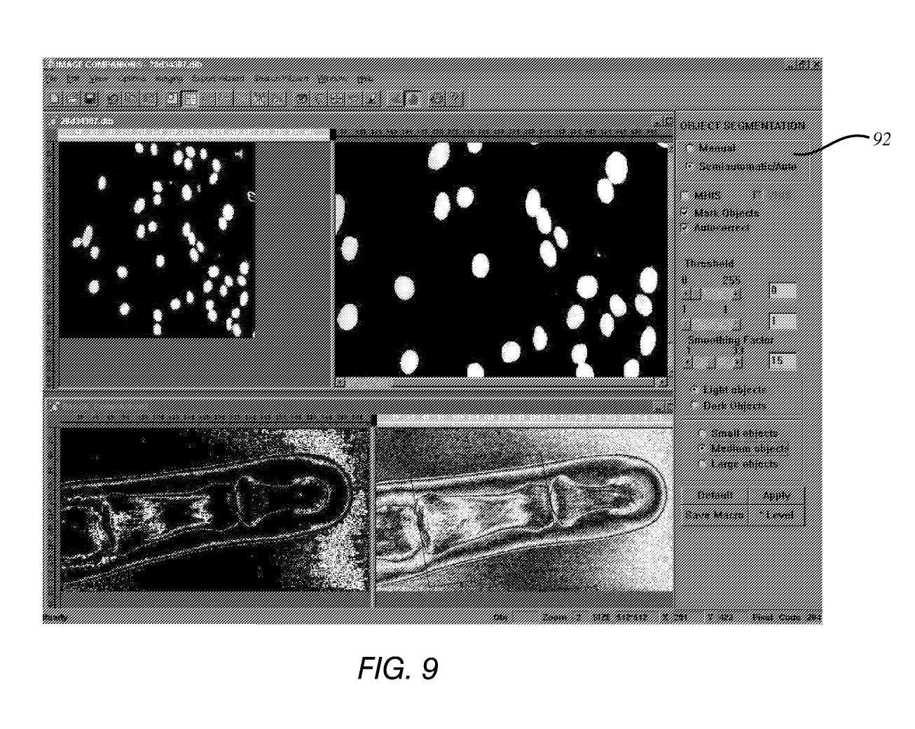

User control of object definition (corresponding to module 34 of FIG. 2) is illustrated in FIG. 9. By selecting one of the checkboxes 92, the user implements manual or semi-automatic object definition. In manual mode, slidebars allow the user to select a brightness range of pixels. All pixels outside this range are considered background. An object is thus defined as a connected set of pixels having brightness values in the user defined range. Background pixels may be reassigned a zero brightness value. In the automatic mode, the user interface for which is illustrated in FIG. 9, the thresholds are calculated automatically by the system from the image histogram. In this mode, the system may allow the user to set up multiple thresholds by setting their values manually or by choosing their sequential numbers from the automatically calculated table of thresholds.

As was the case with the filtering process, the image (or region of interest) is displayed as the object definition function is applied. Those of skill in the art will understand that a wide variety of techniques for assigning pixels to objects or background are known and used, any one of which now known or developed in the future may be used in conjunction with the present invention.

After objects are defined/detected, parameter sets are calculated for each object, and then comparisons are possible to find similar objects (or object clusters as discussed above) in either the same image or in different images. This is illustrated in FIG. 9, which shows a display of the original image 104 after filtering and object segmentation, as well as the template 106 selected for comparison to objects in the remainder of the image. In this example, the template 106 is a three object cluster. Also provided in this screen display are seven displays 108a-g which display in rank order the seven objects of the image most similar to the template object. Also displayed at 110 is a list of the parameters used in the comparison and the weights assigned to them for the comparison process. These weights may be manually set, or they may be set via a statistical process which is described in further detail below.

The actual comparison process which defines the degree of template similarity may, for example, be performed with the following formulas. For templates consisting of one individual parameterized object, a parameter difference vector may be computed which has as each element the difference between the parameter values divided by the maximum difference observed between the template object and all objects being compared to the template.

Δᵢₜ(Pᵢₜ,Pⱼ)/Δmax(Pᵢₜ,Pₖ), (20)
where

P is a parameter-vector; it is the index of template object; k=1, . . . , L; L is all objects that the template object is being compared to; and j is the index of specific object being compared to template object.

A numerical similarity may then be computed using either a modified form of Euclidean or Minkowski line metrics or as modified Voronin formula as set forth below:

$$\{ \begin{matrix}{( {\sum }_{k = 1}^{L} {( {p}_{k}^{t} - {P}_{k}^{t} )}^{s} * {ω}_{k} )}^{1 / s} \\ and \\ {( {P}_{i} - {P}_{k} )}^{T} {W}^{- 1} ( {P}_{i} - {P}_{k} ) , \\ where W is the covariation matrix ; \\ ω is a statistical weight\end{matrix} \\ and in our modification is p = {p}_{k}^{t} / ( max {p}_{k} - min {p}_{k} ) \qquad ( 21 )$$

For multi-object templates or entire images, the spatial relationship between selected objects of the template to other objects in the template may be numerically characterized and effectively added as one or more additional subvectors of the object parameter vector. The overall similarity between a multi-object template and object clusters in the image database, may, in some embodiments of the invention be calculated as follows:

$$ζ = {\sum }_{j = 1}^{Z} ϖ * abs ( {η}_{ij}^{t} ) / Z , \\ where Z - number of components , \\ {η}_{ij}^{t} = 1 - abs ( {Δ}_{i}^{t} - {Δ}_{j}^{t} ) / ( max {Δ}_{t} - min {Δ}_{t} ) , \\ {Δ}^{t} = \{ \begin{matrix}1 , & when abs ( {Δ}_{i}^{t} - {Δ}_{j}^{t} ) \leq {ɛ}_{t} \\ 0 , & else\end{matrix} \qquad ( 22 )$$

ε is a thresholds and/or tolerances vector,

\tilde{ω} is a weights vector

This formula combines not only parametric similarity but spatial similarity also. For spatial similarity the closeness of the position and pattern fit for objects of the template and objects of the database are numerically evaluated. The mathematical method for parameterizing these spatial relationships may, for example, use some simple Euclidean distances between objects for primitive cases and up to pattern fit calculations based on second, third, or fourth moments of inertia for comparable components in complex cases.

Once the objects are parameterized and the template is defined as either a single object or a cluster of objects, the comparison calculation involves the mathematical generation of a value which characterizes how “similar” two vectors or matrices of numbers without further reference to the meaning associated with those numbers. A wide variety of mathematical techniques are available to perform such a numerical characterization, and different approaches may be more suitable than others in different contexts. Thus, the specific formalism used to mathematically define and quantify similarity between number sets may vary widely in different embodiments of the invention and different techniques may be appropriate depending on the application.

As discussed above, the weight assigned to a given parameter during this comparison process may be manually set by the user or set using a statistical method. The statistical method is especially useful when the database of images includes a large number of objects which have been characterized as having or not having a characteristic trait, such as an area of skin pigmentation is either melanoma or not melanoma, or which have been characterized numerically as more similar or less similar to a “model” object. When this data is available, it can be analyzed to determine how strongly different parameters of the parameter set values correlate with the presence or absence of the specific trait.

The weight used for a given parameter in the comparison process may thus be derived from the values of the parameter vectors associated with the detected objects in the image database.

In using this method a system is represented as a totality of factors. The mathematical simulation tools are correlation, regression, and multifactor analyses, where the coefficients of pairwise and multiple correlation are computed and a linear or non-linear regression is obtained. The data for a specific model experiment are represented as a matrix whose columns stand for factors describing the system and the rows for the experiments (values of these factors).

The factor Y, for which the regression is obtained, is referred to as the system response. (Responses are integral indicators but theoretically, any factor can be a response. All the factors describing the system can be successively analyzed.).

The coefficients of the regression equation and the covariances help to “redistribute” the multiple determination coefficient among the factors; in other words the “impact” of every factor to response variations is determined. The specific impact indicator of the factor is the fraction to which a response depending on a totality of factors in the model changes due to this factor. This specific impact indicator may then be used as the appropriate weight to assign to that factor (i.e. parameter of the parameter set associated with the objects).

The impact of a specific factor is described by a specific impact indicator which is computed by the following algorithm:

γⱼ=α*[bⱼ*c₀ⱼ], j=1, 2, . . . , k, (23)

where γ is the specific impact indicator of the j-th factor; k is the number of factors studied simultaneously; bj is the j-th multiple regression coefficient which is computed by the formula

X₀=a+Σbⱼ*Xj, (24)

where X₀ is the system response to be investigated, a is a free term of the regression, and Xⱼ is the value of the j-th factor. The coefficient α of the equation is computed by the formula

α=R²/[Σⱼ|bⱼ*c₀ⱼ|], (25)

where R is the coefficient of multiple determination computed by the formula

R=[(n²*Σⱼbⱼ*c₀ⱼ)/(n*Σⱼx²₀ⱼ−(Σⱼx₀ᵢ)²)]^{1/2}, (26)

where n is the number of observations, which cannot be below (2*K); x₀ᵢ is the value of the system response in the i-th observation, c₀ⱼ is the covariance coefficient of the system response indicator and the j-th factor. It is given by the relation

c₀ⱼ=(n*Σᵢx₀ᵢ*xⱼᵢ−Σᵢx₀ᵢ*Σᵢxⱼᵢ)/n² (27)

The specific contribution indicator is obtained mainly from the coefficient of multiple determination, which is computed by the formula

R²=(Σⱼbⱼ*c₀ⱼ)/D² (28)

where D² is the response variance. The specific impact of the j-th factor on the determination coefficient depends only on the ratio of addends in this formula. This implies that the addend whose magnitude is the largest is associated with the largest specific impact. Since the regression coefficients may have different signs, their magnitudes have to be taken in the totals. For this reason, the coefficients γ of the specific impact are bound to be positive. However, it is important that the direction in which the factor acts by the computed γ is dictated by the sign of the regression coefficient. If this sign is positive, the impact on the response variable is positive and if it is not, the increase of the factor results in a reduction of the response function. The influence of the background factors, which are not represented in the data, is computed by the formula

γ̅ᵢ=1−Σⱼγⱼ. (29)

The importance of the γ is determined from the relation for the empirical value of the Fisher criterion

Fⱼ=(γⱼ*(n−k−1))/(1−Σⱼγⱼ). (30)

A rearrangement of the initial data matrix at every experimental step makes it possible to investigate successively the dynamics of the significance of the impact the factors have on all system indicators that become responses successively. This method increases the statistical significance of the results obtained from the algorithm for the recomputation of the initial data matrix. The algorithm embodies serial repeatability of the experiments by fixing the factors at certain levels. If the experiment is passive, the rows of the initial matrix are chosen in a special way so that, in every computation, rows with the closest values of factors (indicators) influencing the response are grouped together. The dynamics of the specific contributions is computed by using the principle of data elimination.

In the proposed way, the computation of the dynamics of the insignificant information is gradually eliminated. The value of γ does not change remarkably until the significant information is rejected. A dramatic reduction of γ is associated with a threshold with which this elimination of useful information occurs. The algorithm of this operation is an iterative γ recomputation by formula (23) and a rejection of information exceeding the threshold computed. In the algorithm, the significance of the result and of the information eliminated is increased by recomputing the initial data matrix into a series-averaged matrix, the series being, for instance, the totality of matrix rows grouped around the closest values of the factor in the case of a passive factorial experiment. The series may also consist of repeated changes of the indicator with the others fixed at a specified level. Because in further discussion the series-averaged matrix is processed in order to obtain final results, the compilation of series from the data in a field is a major task for the user because, both, the numerical and meaningful (qualitative) result of the computation may be influenced. With increasing threshold the amount of rejected information also increases, therefore one has to check whether the amount of information in the series-averaged matrix is sufficient, see below. Consequently, the information on the factor considered in this version of the method is rejected by the formula

X₁ᵢ=[ΣₚX₁ᵢₚ−m*h]/nᵢ, p=1,2 . . . , m; i=1,2, . . . , N, (31)

where X₁ᵢ is the value of the i-th series in which the factor X₁ is observed and for which the critical (rejection) threshold is determined after the elimination of data with a threshold of H; nⱼ is the number of observations in the i-th series; m is the number of values of the X₁ which exceed h and (0≦m≦nᵢ); N is the number of observation series (rows of the N*(K+1) matrix of the initial information, where K is the number of factors investigated simultaneously.)

The invention thus provides image searching and comparison based in a much more direct way on image content and meaning than has been previously available. In addition, using the described method of weights calculations for targeting similarities between a multi-component template and a database of images in medical fields is much more mathematically justified and sound than neural network techniques used for the same purposes. That is important to understand because template matching may be used in such applications to decrease the difficulty of database creation and search, and improve early cancer diagnostics, early melanoma detection, etc.

As set forth above, diagnosis, assessment or estimation of level of likelihood of potential disease states is facilitated by noting that an object in a query image is or is not similar to objects previously classified as actual examples of the disease state. In some embodiments, diagnosis, assessment or level of likelihood of potential disease states is facilitated by computing a numerical score which is an assessment or is indicative of the likelihood that a particular diagnosis (e.g. malignant melanoma or benign growth, benign breast lesion or carcinoma) or biomedical or physical condition is correct. This score may be computed based on or using an analysis of the numerical similarity and features computations between or of an object or objects in the query image and previously classified or assessed objects in the database. Several new methods that advanced the scoring computations based on diagnostic findings or condition assessment are proposed as set forth below.

Algorithm 1: This is a first order ranking method, essentially a binary classification of the query object. The software calculates and retrieves the T_{ψ} closest matches in the database to the unknown object. The database objects were previously detected, defined and quantified. Then the rank is assigned according to a rule: if more than a half of the closest template objects T_{ψ} have been diagnosed or assessed as no disease then the score for the unknown object shall reflect no disease finding, otherwise the score reflects disease or its likelihood.

Algorithm 2. This is a simple Averaging Ranking Scoring system. Continuum similarity values for the closest T_{ψ} templates objects with known findings are substituted by their dichotomic ranks (e.g. −1 for benign or 5 for malignant, or 1 for presence of the disease and 0—for its absence). Then the assigned score is an average of the T_{ψ} ranks.

Algorithm 3. Scoring with the penalty function. The method uses only the maximum number Tτ of closest templates objects that corresponds to the highest ranking value τₘₐₓ in the scoring range. The values of calculated similarities between each template with known finding and the unknown object is substituted with the values that are calculated as follows:

For Templates of highest τₘₐₓ:

τₘₐₓ−Penalty*Relative Similarity; (32)

For Templates of τₘᵢₙ:

τₘᵢₙ+Penalty*Relative Similarity.

For example, if τₘₐₓ is equal 5 and τₘᵢₙ is equal 1 and the Relative Similarity based retrieved closest matches for cluster of 6 are (62.24% 60.78% 60.48% 59.68% 59.49% 59.23%) with diagnostic findings as follows (benign malignant benign benign benign benign maligant) then the score for. i.e. second template in the cluster will be equal to 5+(5−1)*(60.78−100)/100=3.431.

Algorithm 4. Averaging with weights for position with fixed retrieved templates cluster method. The software calculates and retrieves the T_{ψ} closest matches to the unknown object that represents the manifestation of the disease (i.e. lesion, skin growth, etc). These objects were detected, defined and quantified. Continuum similarity values for the closest T_{ψ} templates objects with known findings are substituted by their dichotomic ranks (i.e. −1 for benign or 5 for malignant, or 1 for presence of the disease and 0—for its absence). Then the assigned score is an average of the T_{ψ} ranks, however each rank is multiplied by the evenly distributed weight calculated for its position in retrieved cluster. Each weight can be calculated in different ways—for example as follows: for each position above the middle position of the cluster the current rank gets its weight increased by 1, for every position below the middle position of the cluster the current rank gets its weight decreased by 1 (i.e. if the cluster N_{c} is 7 then the score of the closest T_{ψ} template object will have its weight of (7+1+1+1)/7=10/7. In other words if we have the following sequence of the closest matches malignant-benign-benign-malignant-malignant-benign-malignant in N_{c}=7 templates cluster and malignant is indicated by the score 5 and benign is indicated by the score 2 then the calculated total score will be

(5*10/7+2*9/7+2*8/7+5*7/7+5*6/7+2*5/7+5*4/7)/7=3.653).

Algorithm 5. Averaging with weights for position method with floating retrieved templates cluster method. The method is similar to Algorithm 4 except number N_{c} of templates in each retrieved cluster is truncated. The truncation could be done by setting Relative Similarity threshold to, say, 80% or 90%. This way all templates with Relative Similarity below the threshold will not be considered and the value of N_{c} will not be constant like in Algorithm 4.

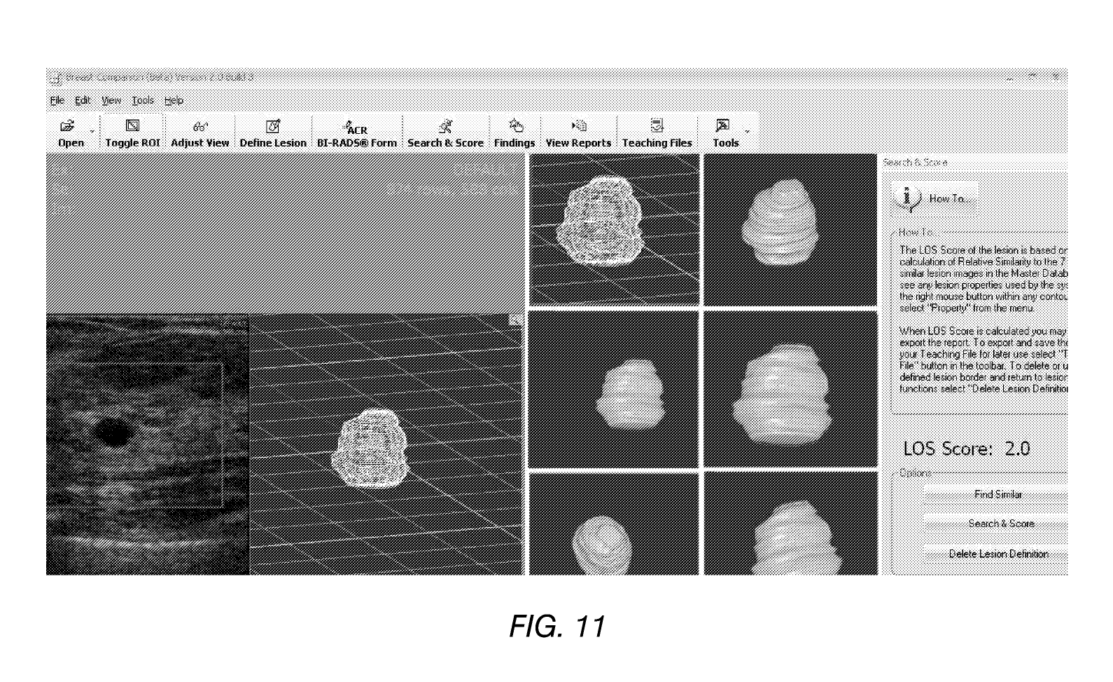

In the example of FIG. 11, existing multiple slices of 3D ultrasound image of a breast lesion were processed by the system, segmented and the selected few scored against digital database of templates with known findings. The result of the database search, retrieval and scoring was displayed in a form of 7 closest matches found and overall score is produced (in our case 2—benign) by one of the five scoring methods described herein below. Then the system rendered 3D image of the processed lesion slices facilitating further quantification of the lesion such as analyses of volume, vortex as well as estimations of the texture and curvature of the lesion surface. It is possible to compare and quantify relative similarity not only individual slices of the lesion but also the rendered 3D lesion or mass as a whole object.

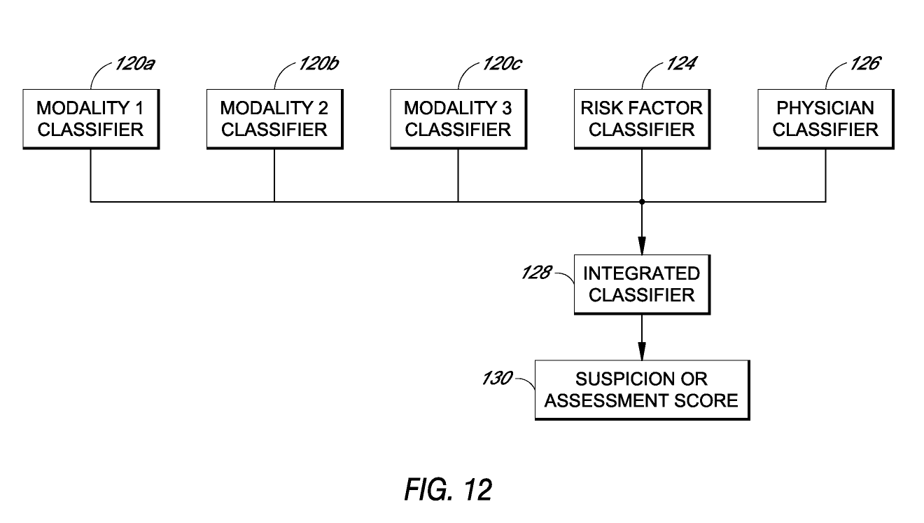

Returning now to a discussion of multimodality fusion analysis, it can also be useful to combine single or multi-modal image analysis with other types of information in order to further refine the resulting score and diagnosis. FIG. 12 illustrates one such embodiment.

Referring to FIG. 12, one or more image acquisition modalities 120a, 120b, and 120c are used to analyze images of the suspected lesion or mass as described above with reference to FIG. 1. In addition, non-image data is used to produce additional numerical classification scores that indicate disease likelihood or assessment. These additional scores may be related to risk factors 124 such as age or other anthropomorphic and biometric information, or demographic profile of the subject of the image, or analysis of behaviors such as smoking, cancer history in the family, race statistical probabilities, genetic statistics, etc. As another alternative, a classification score or assessment from a physician 126 may be generated and utilized. This classification score or assessment may be based on any clinical observations from, for example, the attending physician that can be expected to correlate either positively or negatively with the observed features of the image and object in question in the image and/or with the presence of disease. The physician may, for example, make an initial assessment of the patient to get their impressions of the patient condition or patient's clinical history. The numerical classifications from each of the multiple modalities are input to an integrated classification system 128 that combines (fuses) the multiple numerical classifications into a single suspicion score or numeric assessment 130.

In this embodiment, it is especially advantageous to have an integrated classification method that can incorporate inputs from a wide variety of information sources in a consistent and easy manner. There are a variety of “white box” approaches for multiple classifier inputs integration (compare to “black box” approached such as Artificial Neural Networks, Classic Regression, Bayesian Statistics, etc). One such “white box” approach was modified as set forth below to incorporate statistical weighting function (see formula (23) above) that can be used is as follows:

For the sake of this text we will use terms Computerized Lesion Assessment (CLA) or in more generic term Level of Suspicion (LOS) as the numerical classifier indicating some estimate of disease likelihood, an initial assessment of the condition by a practitioner, etc. Let S denote a set of diagnoses. The LOS, represented by m, defines a mapping of the power set P(S) (set of all subsets of S) to the normalized interval between 0 and 1. Apportions ‘mass’ or weight of evidence to each subset. The sum of LOS's over all subsets must equal one.

Belief or Fusion function for a set A is defined as the sum of all the Level of Suspicion Assessments of the subsets B of A:

$$Bel ( A ) = {\sum }_{B \vert B ⋐ A} m ( B ) , \qquad ( 33 )$$

Where m(B) can be modified by statistical weight γⱼ computed according to (23).

The Dempster-Shafer Rule for an integrated classifier can be defined as:

$${m}_{12} ( A ) = \frac{{\sum }_{B ⋂ C = d} {m}_{2} ( B ) {m}_{2} ( C )}{1 - K} when A \neq , \qquad ( 34 )$$

where m₁₂ is the Dempster-Shafer Combination of Mass m₁ of Classifier 1 (Sensor 1); Mass m₂ of Classifier 2 (Sensor 2); K is a normalization factor and can be calculated as

$$where K = {\sum }_{B ⋂ C =} {m}_{1} ( B ) {m}_{2} ( C ) \qquad ( 35 )$$

For diagnostic testing we can describe each case of assessment as S={M1, M2, B}, where M1=Level of Suspicion to cancer Type 1, M2=Level of Suspicion to cancer Type 2, B=Benign. Power Set is a set of all Subsets of S, then:

{M1} Level of Suspicion to cancer Type1

{M2}=Level of Suspicion to cancer Type2

{B}=Benign

{M1, M2}=Level of Suspicion to cancer Type1 or Level of Suspicion to cancer Type2

{M1, B}=Level of Suspicion to cancer Type1 or Benign {M2, B}=Level of Suspicion to cancer Type2 or Benign

{M1, M2, B}=No knowledge

Φ=Neither suspicious to Malignancy nor Benign (Normalizing Factor)

The Bel(A) function can be reformulated as:

Bel({M1,M2,B})=m({M1,M2,B})+(m({M1,M2})+m({M1,B})+m({M2,B})+m({M1})+m({M2})+m({B})

As one example of such a calculation, let say our classifiers produced the following numeric results:

Classifier 1.

m₁({M1})=0.6 or LOS for {M1} is 0.6

m₁({M1,M2})=0.28 or LOS for {M1,M2} is 0.28
Remaining ‘mass’ or LOS is assigned to all other possibilities, indicated by {S} m₁({S}=0.12

Classifier 2.

m₂({B})=0.9 meaning that the LOS for Benign is 0.9

Remaining ‘mass’ is assigned to the remaining possibilities m₂({S})=0.1. Then each Classifiers' fusion could be represented by a set of tables. Each table entry is the product of the corresponding mass values. The intersection of {M1} and {B} is empty, designated by the symbol {Φ}. The intersection of the full set {S} with any set {A}={A}.

| | | m1 | | |
|---|---|---|---|---|
| | | {M1} | {M1, M2} | {S} |
| | | 0.6 | 0.28 | 0.12 |
| | {B} | {Φ} | {Φ} | {B} |
| | 0.9 | 0.54 | 0.252 | 0.108 |
| m2 | | | | |
| | {S} | {M1} | {M1, M2} | {S} |
| | 0.1 | 0.06 | 0.028 | 0.012 |

Dempster-Shafer Normalization Factor is derived from the mass values of the empty sets {Φ} in the table. These empty sets correspond to conflicting evidence from the two sensors. Mass for {Φ}=0.540+0.252=0.792. Normalization factor=1−mass of {Φ}=1−0.792=0.208. Now we can calculate fusion of Classifier 1 and Classifier 2. Each Term is divided by the Normalization Factor 0.208

m₁₂{B}=0.108/0.208=0.519

m₁₂{M1}=0.06/0.208=0.288

m₁₂{M1,M2}=0.028/0.208=0.135

m₁₂{S}=0.012/0.208=0.057

LOS before fusion was:
Classifier 1: Level of Suspicion to cancer Type1=0.6
Classifier 2: Benign=0.9

As a result LOS after fusion is adjusted to:
Level of Suspicion to cancer Type1=0.288
Benign=0.519

It was discovered by us that the accuracy of Dempster-Shafer Normalization Factor can be increased by applying statistical weights from calculated using formula (23) above to calculation in formula (33) for Bel(A).

One important aspect of the above described method is that it does not matter what the source of the input classifications is. It could be from object comparison in an image as described above (e.g. used as the integrated classifier of FIG. 1), it could be a demographic risk factor derived value, or a physician input value (e.g. used as the integrated classifier of FIG. 12). Another advantage of the above method is that the integrated classification score will be highly dependent on consistency and contingency of the input classifications which is intuitively desirable. Other integration methods may be used, preferably sharing the above described advantages.

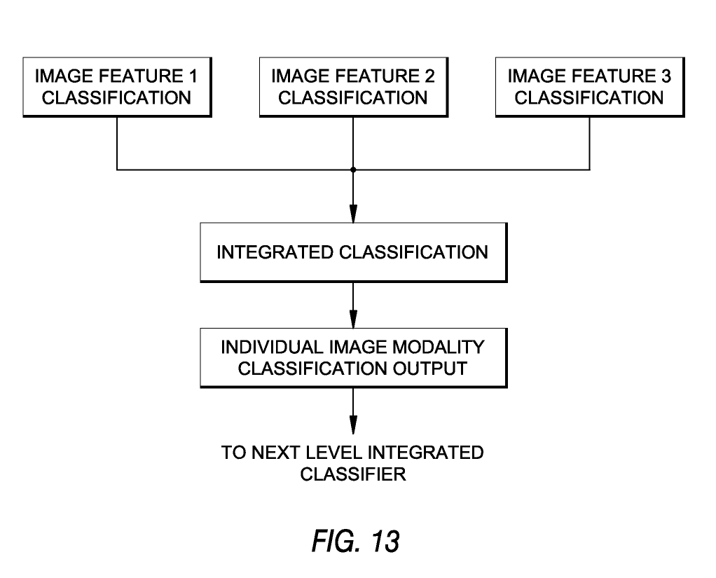

FIGS. 13, 14, and 15 further illustrate the flexibility of this approach in that hierarchies of integrated classification can be created. FIG. 13 shows how classification or assessment scores derived from individual features can be integrated to produce a single image modality classification score that is then input to the next level of integrated classifier such as 114 or 128 of FIGS. 1 and 12. In this embodiment, individual image features or combinations of features such as form factor, optical density, etc. discussed above can be used to produce a score. Separate images can also be used to produce separate scores. These scores are then integrated as described above with reference to FIGS. 1 and 12 (or with another method) to produce a selected image modality classification output (e.g. ultrasound image modality classification output). More detailed method and calculation examples of computation of classification scores from an image or one or more image features are described herein above in paragraphs 0087 through 0092.

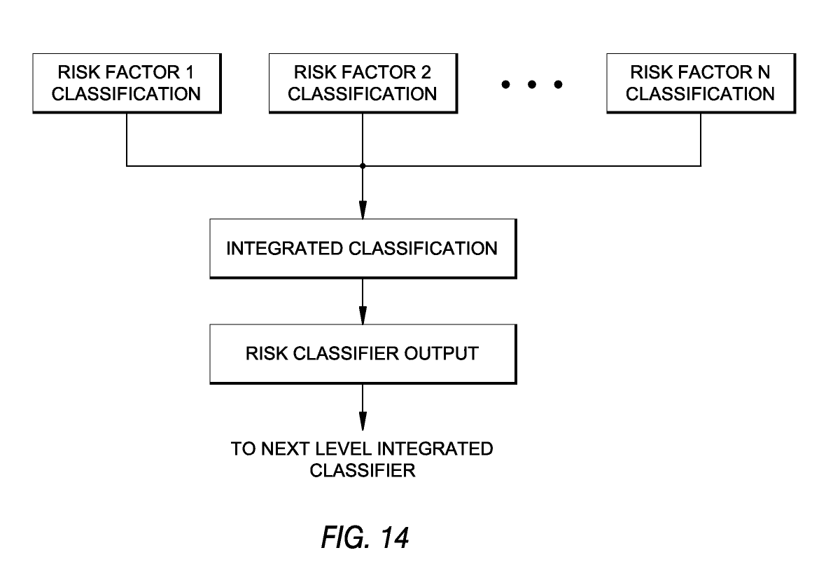

FIG. 14 illustrates the same principle with the risk factor classification score of FIG. 12. Scores produced from different risk factors can be separately generated and then integrated into a risk factor score that may be then input into the next level integrated classifier with other scores from other sources.

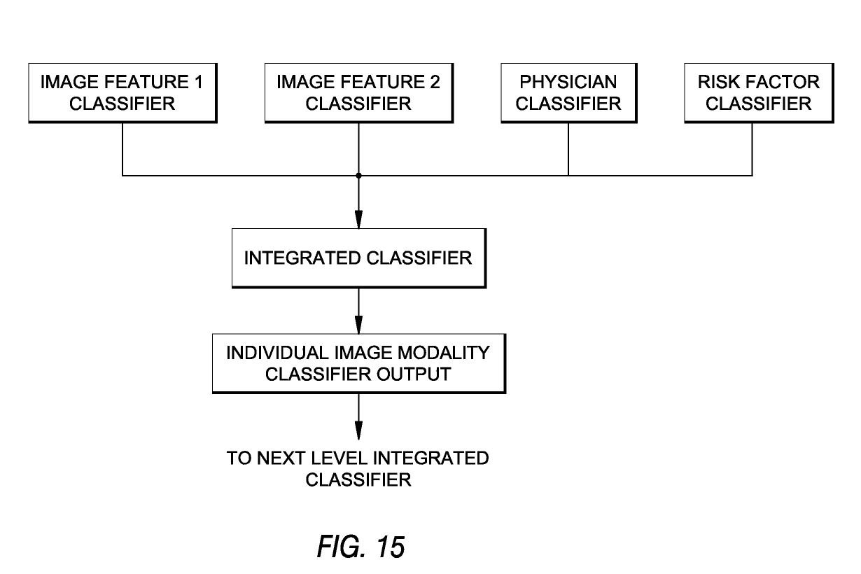

FIG. 15 illustrates that non-image information can be integrated with image information to produce an integrated image modality score that includes information beyond solely image information. This may be useful when certain non-image factors are highly relevant to information received via one image modality and not particularly relevant to information received from other image modalities. In this case, scores or assessment from relevant non-image factors can be integrated into a final score or assessment for the particular image modality for which those factors are relevant.

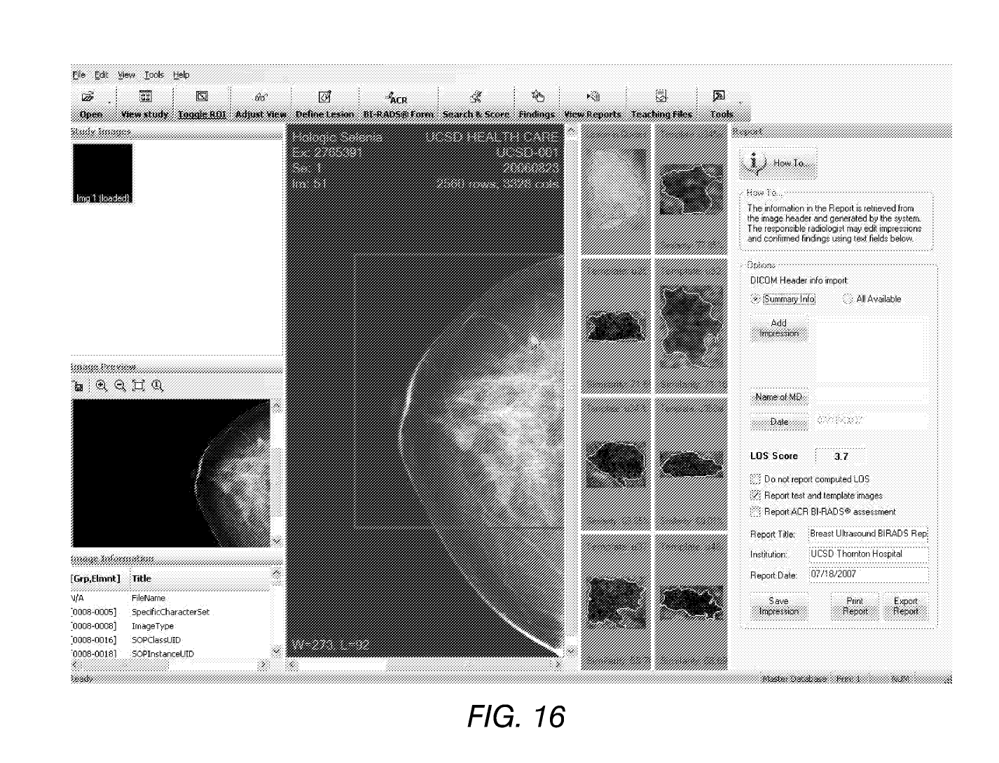

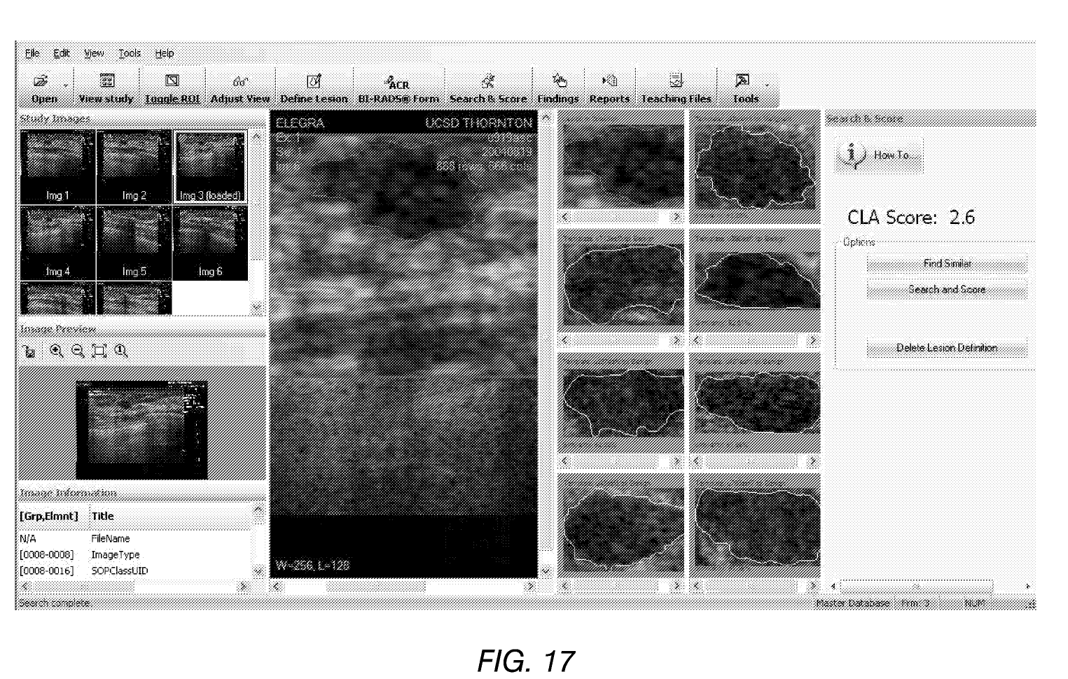

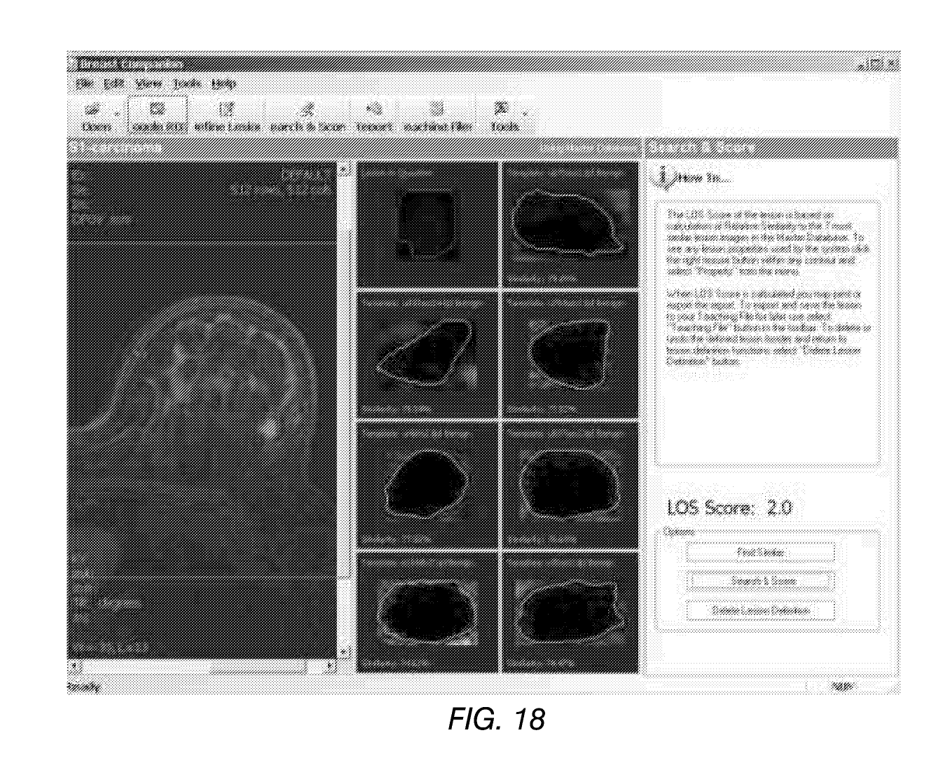

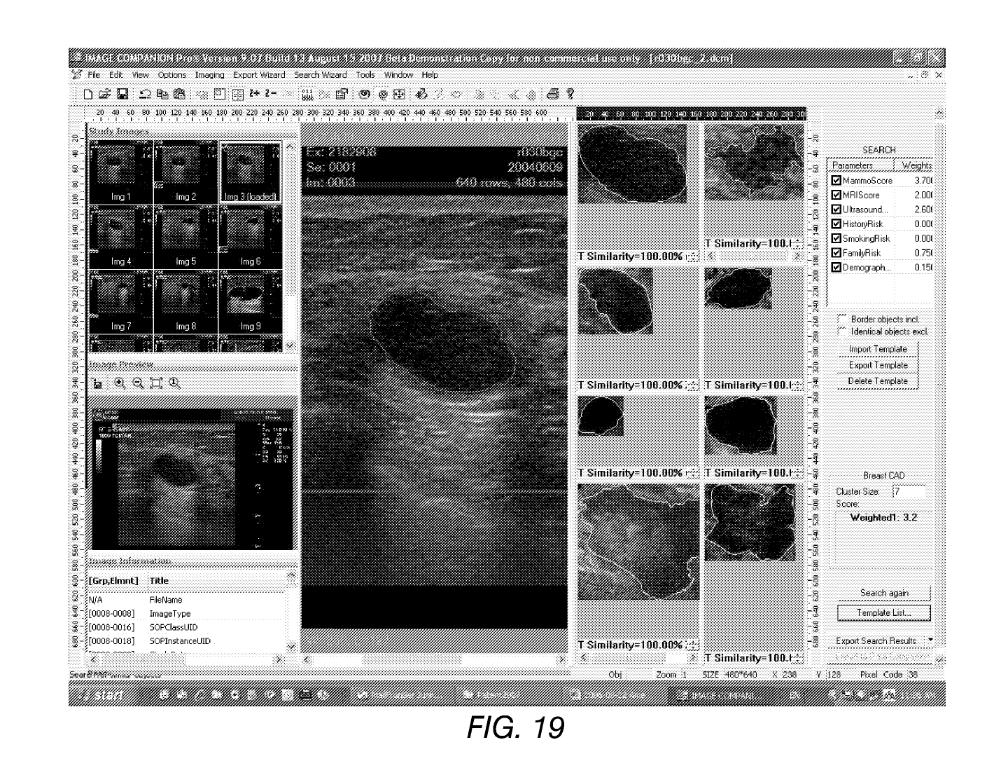

FIGS. 16 through 19 illustrate implemented multi-modality fusion classification. FIG. 16 illustrates an individual score or assessment produced during assessment of breast mammography. FIG. 17 illustrates an individual score or assessment (term Computerized Lesion Assessment or “CLA” is used as more specific analog of generic LOS tem used for non-lesion based diseases or conditions) produced during assessment of breast ultrasound for the same lesion (object). FIG. 18 illustrates an individual Level of Suspicion score or assessment produced during assessment of breast MRI for the same lesion (object). FIG. 19 illustrates multi-modality fusion classification with a variety of risk and demographic factors integrated with output individual classification scores from all three breast related modalities: mammography, ultrasound, MRI. In this final screenshot of multi-step fusion process the system integrated scores from each contributing modalities (mammography 3.7, ultrasound 2.6 and MRI 2.0) with history, family and other risk factors. As it is illustrated despite the fact that all diagnostic modalities—ultrasound and MRI—indicate benign assessment of this lesion (score about 2.0)—Family and Demographic Risks outweigh these computed assessment and the final weighted fusion score using modified Dempster-Shafer Normalization Factor is computed as 3.2—which for breast cancer assessment guidelines means “probably benign, close follow up recommended”)—otherwise would be assessed as—“benign, no suspicion”.

When classification assessment is completed the system may allow the user to display, sort, update and use his/her own Teaching File that consists of already read and confirmed cases. The custom Teaching File consists of images previously processed by radiologists, their associated numeric reporting descriptors and specific to the modality lexicon based descriptors, written impressions and biopsy proven findings. The system allows the user to sort and display confirmed cases from a custom Teaching File based on information contained in the DICOM header of the stored images (that may include such DICOM tags as “diagnostic code”, “date of the examination”, “biopsy date”, keywords in pathology and/or impressions and image features such as dimensions, area, etc.) or modality specific assessment descriptors selected by the radiologist in the modality specific assessment diagnostic or assessment classification form. The user capability of displaying the similar cases together with their impressions, descriptors and pathology is a very valuable educational and training tool proven to be very successful in women's health.

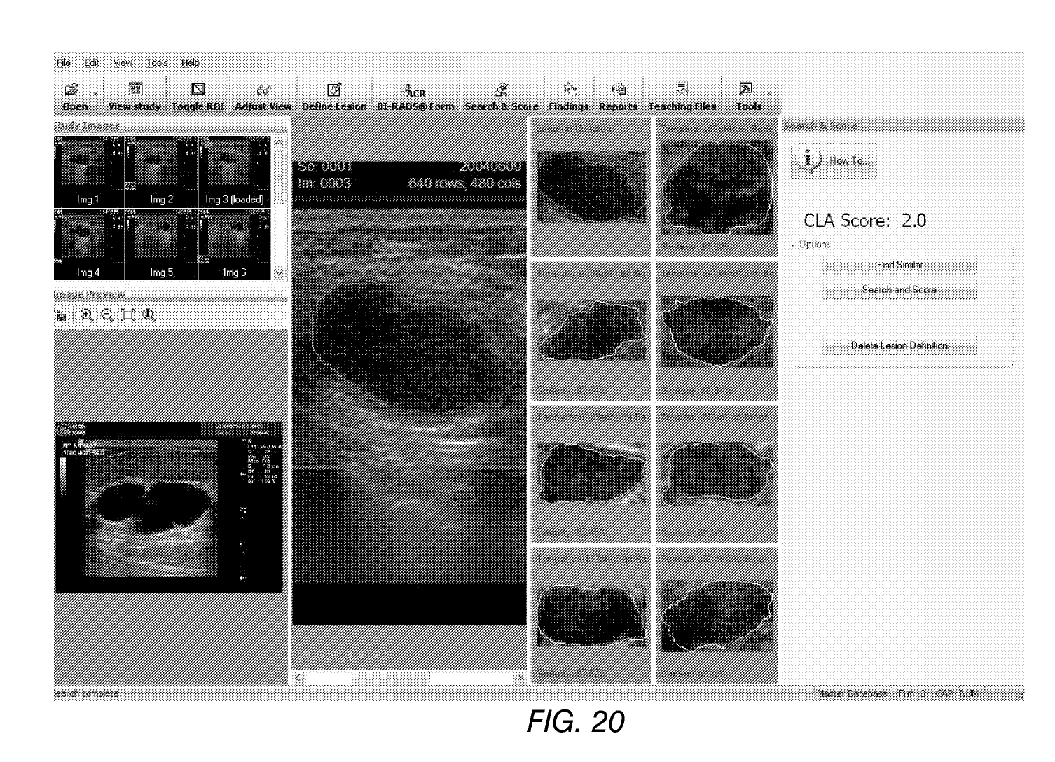

FIG. 20 illustrates one implemented variant of a multimodality Teaching File. The system allows the user to display all images in the case or to select one particular study image for a zoomed view (upper right corner of a set of study images is selected in FIG. 20). It also allows the user to select and view other cases of the same or different modalities with confirmed findings from the Teaching File, PACS, or other digital image sources. DICOM tags of all viewed images are displayed in the lower left corner.

It is advantageous in a multimodality system that the Teaching File be able to handle each modality separately as well as provide a way to input and save impressions, descriptors, etc. for the fused classification scoring as well. In the context of practical clinical use, automated computerized image analysis and diagnostic tools are most useful when physicians and other users of the system can annotate processed cases and search for cases previously processed for both single and multiple modality image processing.

The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated. The scope of the invention should therefore be construed in accordance with the appended claims and any equivalents thereof.

What is claimed is:

1. A computer implemented method of producing a disease assessment, said method comprising:
producing a first numerical disease or condition classification or assessment score from at least one image;
producing a second numerical disease or condition classification or assessment score from non-image information;
combining at least the first and second disease or condition classification or assessment scores to produce a combined disease or condition classification or assessment score; and
displaying the combined disease or condition classification or assessment score.

2. The method of claim 1, wherein the non-image information comprises demographic information.

3. The method of claim 1, wherein the non-image information comprises age and other anthropomorphic and biometric information.

4. The method of claim 1, wherein the non-image information comprises risk information.

5. The method of claim 1, wherein the non-image information comprises at least one physician diagnosis or impression.

6. The method of claim 1, wherein said first disease or condition classification or assessment score is derived at least in part by comparing an object in a first image with objects in other images.

7. The method of claim 1, comprising:
producing a third numerical disease or condition classification or assessment score from additional image information;
combining at least the first, second, and third disease or condition classification or assessment scores to produce a combined disease or condition classification or assessment score.

8. The method of claim 7, wherein the additional image information is derived from different image modalities from the first image information.

9. The method of claim 1, wherein the combined disease or condition classification or assessment score is dependent on the consistency and contingency between the first and second disease or condition classification or assessment scores.

10. The method of claim 9, wherein the combined disease or condition classification or assessment score is produced with a modified Dempster-Shafer normalization factor.

11. The method of claim 1 wherein one or both of the first and second disease or condition classification or assessment scores comprise combined classification scores.

12. The method of claim 1, additionally comprising storing said first, second, and combined disease or condition classification or assessment scores in a teaching file in associate with physician input information.

13. A computer implemented method of producing a disease suspicion score, said method comprising:
producing a first numerical disease or condition classification or assessment score from at least one image produced with a first imaging modality;
producing a second numerical disease or condition classification or assessment score from at least one image produced with a second imaging modality;
combining at least the first and second disease or condition classification or assessment scores with non-neural network statistical analysis to produce a combined disease or condition classification or assessment score; and
displaying the combined disease or condition classification or assessment score.

14. The method of claim 13, wherein the combined disease or condition classification or assessment score is dependent on the consistency between the first and second disease or condition classification or assessment scores.

15. The method of claim 14, wherein the combined disease or condition classification or assessment score is produced with a modified Dempster-Shafer normalization factor.

16. The method of claim 13, additionally comprising storing said first, second, and combined disease or condition classification or assessment scores in a teaching file in associate with physician input information.

17. A system for producing a disease assessment, said system comprising:
means for producing a first numerical disease or condition classification or assessment score from at least one image;
means for producing a second numerical disease or condition classification or assessment score from non-image information; and
means for combining at least the first and second disease or condition classification or assessment scores to produce a combined disease or condition classification or assessment score.

18. The system of claim 17, wherein both means for producing and the means for combining comprise software modules stored in a computer readable memory.

19. A system for producing a disease suspicion score, said system comprising:
means for producing a first numerical disease or condition classification or assessment score from at least one image produced with a first imaging modality;
means for producing a second numerical disease or condition classification or assessment score from at least one image produced with a second imaging modality; and
means for combining at least the first and second disease or condition classification or assessment scores with non-neural network statistical analysis to produce a combined disease or condition classification or assessment score.

20. The system of claim 19, wherein both means for producing and the means for combining comprise software modules stored in a computer readable memory.
